(19) United States
(12) Patent Application Publication
(10) Pub. No.: US 2026/0256685 A1
(43) Pub. Date: Sep. 3, 2026

(54) COSMETIC SKIN FORMULATIONS AND METHODS OF USING THEREOF

(71) Applicant: Olaplex, Inc., Santa Barbara, CA (US)

(72) Inventors: Lavinia POPESCU, New York, NY (US); Yuxuan LIANG, New York, NY (US); Gloria LABARCA, New York, NY (US); Emre DOGU, New York, NY (US)

(21) Appl. No.: 19/547,743

(22) Filed: Feb. 24, 2026

Related U.S. Application Data
(63) Continuation of application No. 19/067,473, filed on Feb. 28, 2025, now abandoned.

(51) Int. Cl. A61K 8/9789; A61K 8/34; A61K 8/44; A61K 8/49; A61K 8/99; A61Q 19/08

(52) U.S. Cl. CPC A61K 8/9789; A61K 8/345; A61K 8/447; A61K 8/4913; A61K 8/99; A61Q 19/08; A61K 2800/85

(57) ABSTRACT

Cosmetic skin formulations, as well as methods of using thereof are described herein. The cosmetic skin formulations provide a beneficial effect and/or an improvement to the appearance and function of skin when applied thereto.

### CROSS-REFERENCE TO RELATED APPLICATION

This application is a continuation of U.S. application Ser. No. 19/067,473 filed Feb. 28, 2025, the entire disclosure of which is hereby incorporated by reference.

### FIELD OF THE INVENTION

The present invention generally relates to cosmetic skin formulations, as well as methods of using thereof.

### BACKGROUND OF THE INVENTION

A variety of formulations for skin have been developed to date. Such skin creams are topical formulations which are applied to skin for various purposes, including moisturizing, nourishing, protecting, treating, and/or preventing specific skin concerns or conditions. Such skin creams can be used, for instance, as moisturizing creams, anti-aging creams, sensitive skin creams, and barrier repair creams. These are just a few examples of the types of commercially available skin creams.

In spite of the many types of available skin formulations, there is still a need for improved skin formulations.

Therefore, it is an object of this invention to provide cosmetic skin formulations which provide improved benefits to skin.

It is still a further object of this invention to provide methods of using such cosmetic skin formulations.

### SUMMARY OF THE INVENTION

Various cosmetic formulations for skin and methods of using thereof are described herein. In a non-limiting instance, a cosmetic skin formulation includes:
(i′) a first composition including glycerin and Lavandula angustifolia extract;
(ii′) at least one of:
   (a) a second composition including Vibrio alginolyticus ferment filtrate;
   (b) a third composition including acetyl tyrosine, proline, and adenosine triphosphate;
   (c) cetylhydroxyproline palmitamide;
   (d) N-Succinyl-S-Famesyl-L-Cysteine;
(iii′) one or more dermatologically acceptable carriers; and
(iv′) optionally one or more dermatologically acceptable excipients.

In another non-limiting instance, a cosmetic skin formulation includes:
(i″) a first composition including glycerin and Lavandula angustifolia extract;
(ii″) N-Succinyl-S-Farnesyl-L-Cysteine; and
(iii″) optionally at least one of:
   (a) a second composition including Vibrio alginolyticus ferment filtrate;
   (b) a third composition including acetyl tyrosine, proline, and adenosine triphosphate;
   (c) cetylhydroxyproline palmitamide;
(iv″) one or more dermatologically acceptable carriers; and
(v″) optionally one or more dermatologically acceptable excipients.

In yet another non-limiting instance, a cosmetic skin formulation includes:
(i′″) a first composition including glycerin and Lavandula angustifolia extract;
(ii′″) cetylhydroxyproline palmitamide; and
(iii′″) optionally at least one of:
   (a) a second composition including Vibrio alginolyticus ferment filtrate;
   (b) a third composition including acetyl tyrosine, proline, and adenosine triphosphate;
   (c) N-Succinyl-S-Farnesyl-L-Cysteine;
(iv′″) one or more dermatologically acceptable carriers; and
(v′″) optionally one or more dermatologically acceptable excipients.

In still another non-limiting instance, a cosmetic skin formulation includes:
(i″″) a first composition including glycerin and Lavandula angustifolia extract;
(ii″″) a second composition including Vibrio alginolyticus ferment filtrate; and
(iii″″) optionally at least one of:
   (a) a third composition including acetyl tyrosine, proline, and adenosine triphosphate;
   (b) N-Succinyl-S-Farnesyl-L-Cysteine;
   (c) cetylhydroxyproline palmitamide;
(iv″″) one or more dermatologically acceptable carriers; and
(v″″) optionally one or more dermatologically acceptable excipients.

In another non-limiting instance, a cosmetic skin formulation includes:
(i′″″) a first composition including acetyl tyrosine, proline, and adenosine triphosphate;
(ii′″″) at least one of:
   (a) a second composition including Vibrio alginolyticus ferment filtrate;
   (b) a third composition including glycerin and Lavandula angustifolia extract;
   (c) cetylhydroxyproline palmitamide; and/or
   (d) N-Succinyl-S-Famesyl-L-Cysteine;
(iii′″″) one or more dermatologically acceptable carriers; and
(iv′″″) optionally one or more dermatologically acceptable excipients.

In still another non-limiting instance, the cosmetic skin formulation is a scalp formulation that includes:
(1′) at least one active agent of Formula I (as described below); and a
(2′) a first composition comprising glycerin and Lavandula angustifolia extract;
(3′) optionally one or more dermatologically acceptable excipients; and
(4′) one or more dermatologically acceptable carriers.

In some particular instances, the active agent of a scalp formulation is bis-aminopropyl diglycol dimaleate, which can be represented by the following chemical structure:

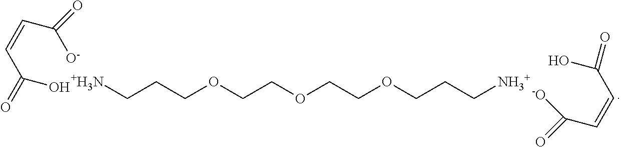

The cosmetic skin formulations disclosed herein are applied to the skin of a subject, such as a mammal (typically a human). The cosmetic skin formulations typically include a combination of agent(s), composition(s), and other optional component(s). It is understood that the skin formulation is a blend of these agent(s), composition(s), and other optional component(s) and typically forms a homogeneous composition thereof. In some instances, the cosmetic skin formulations can include different compositions, such as:
a composition including glycerin and Lavandula angustifolia extract;
a composition including Vibrio alginolyticus ferment filtrate; and/or
a composition including acetyl tyrosine, proline, and adenosine triphosphate.

The cosmetic skin formulations can also include other agents/components, such as:
cetylhydroxyproline palmitamide and/or N-Succinyl-S-Farnesyl-L-Cysteine.

The cosmetic skin formulations typically include one or more dermatologically acceptable carriers. Such carriers can be water, dermatologically acceptable organic solvents, or a combination thereof.

In some instances, the cosmetic skin formulation includes one or more dermatologically acceptable excipients. For example, the dermatologically acceptable excipient can be an abrasive, an acidulent, an adhesion promoter, an antioxidant, an antiseptic, an astringent, a barrier agent, a bio-adhesive agent, a botanical agent, a botanical extract, a buffer agent (such as lactic acid, sodium lactate, citric acid, trisodium citrate, sodium hydroxide), a bulking agent, a calming agent, a chelating agent (such as EDTA, tetrasodium glutamate diacetate), a cleansing agent, an emulsifier agent, a colorant, a conditioning agent, a cooling agent, a deodorant agent, a depilatory agent, a detergent, a disinfectant, a dispersant (such as hydroxyethyl acrylate/sodium acryloyldimethyl tauronate copolymer), an emollient, an emulsifier, an emulsion stabilizer, an essential oil, an exfoliant, a fat soluble agent, a fiber, a film former, a fixative, a foam booster, a foam stabilizer, a foaming agent, a fragrance, a free radical scavenger, a fungicide, a gellant, a glosser, a conditioning agent, a humectant, a lathering agent, a lubricant, a make-up agent (such as to provide a color, tint, gloss, or matte to the formulation and/or the skin to which it is applied), a moisture barrier agent, a moisturizing agent, a neutralizer, an odor-masking agent, an oil, an oil absorbent agent, an ointment base, an opacifier, an organosilicone, an oxidant, an oxygen carrier, a pearlant agent, a perfume, a perfume solvent, a perfume stabilizer, a peroxide stabilizer, a photo-sensitizer agent, a pigment, a pigmenting agent, a pearlescent aid, a plant extract, a plant derivative, a plant tissue extract, a plant root extract, a plant seed extract, a plant oil, a plasticizer, a polymer, a polymer film former, a preservative agent, a propellant, a reducing agent, a re-fatting agent, a resin, a scalp agent, a scrub agent, a sequestrant, a silicone agent, a skin clarifier, a skin cleanser, a skin lipid, a skin lightening agent, a skin bleaching agent, a solubilizer, a suspending agent, a sun protection agent, a soothing agent (such as bisabolol), a spreading agent, a stabilizer, a sunless tanning agent, a sunscreen, a UVA sunscreen, a UVB sunscreen, a broad-band sunscreen, a surfactant, an amphoteric surfactant, an anionic surfactant, a cationic surfactant, a non-ionic surfactant, a silicone surfactant, an accelerator, a thickening agent, a thixotrope, a tightening agent, a toner, a tonic agent, a vegetable oil, a volatile agent, a viscosity stabilizer, a vitamin, a water proofing agent, a wax, a wetting agent, a whitening agent, or a combination thereof.

In some instances, the cosmetic skin formulations can further include one or more additives. For example, the additive can be an agent modulating cell differentiation, an agent modulating cell proliferation, an agent stimulating synthesis of dermal or epidermal macromolecules, an agent preventing degradation of dermal or epidermal macromolecules, an agent acting on microcirculation, an antimicrobial sequestering agent, an analgesic, an anesthetic, an anti-acne agent, an anti-aging agent, an anti-wrinkle agent, an anti-atrophy agent, an anti-bacterial agent, an anti-scar agent, an anti-seborrheic agent, an anti-cracking agent, an anti-cellulite agent, an anti-stretch mark agent, an anti-dandruff agent, an anti-fungal agent, an antihistamine agent, an anti-inflammatory agent, an anti-irritant agent, an anti-microbial agent, an antibiotic agent, an antiviral agent, an anti-skin cancer agent, an anti-eczema agent, an anti-psoriasis agent, an antiperspirant agent, an anti-pruriginous agent, an anti-pruritic agent, a circulatory stimulant agent, a collagen stimulating agent, an elastin stimulating agent, an extracellular matrix stimulating agent, an enzyme, an enzymatic inhibitor, an enzyme-inducing agent, a hormone, a hormone-like agent, a hyaluronic acid stimulating agent, a keratolytic agent, a muco-adhesive agent, a natural moisturizing agent, a lipolytic agent, a peptide agent (such as palmitoyl tripeptide-1, palmitoyl tetrapeptide-7), a protein agent, a rosacea inhibitory agent, a scar prevention agent, a skin barrier agent, a skin barrier restoration agent, a skin calming agent, a skin smoothing agent, a skin calming agent, a skin soothing agent (such as beta-glucan), a skin penetration enhancing agent, a skin penetration retarding agent, a skin conditioning agent, a skin exfoliating agent, a skin peeling agent, a skin healing agent, a skin protectant agent, a skin purifier agent, a vasoconstrictor agent, a wound healing agent, or a combination thereof.

In some instances, the cosmetic skin formulation is a serum, such as a face serum. In some other particular instances, the cosmetic skin formulation is a skin moisturizer formulation.

In some instances, the cosmetic skin formulation is a scalp formulation.

The cosmetic skin formulations described herein may be provided as part of a cosmetic kit.

The cosmetic skin formulations described herein can be used to treat skin. In one non-limiting instant, a method for treating skin includes:
(1) applying a cosmetic skin formulation described to the skin of a subject.

Methods and processes for testing and evaluating the effect(s) and efficacy of a cosmetic skin formulation are known in the field of skin care. For instance, maintaining skin, the prevention, amelioration, and/or treating of the signs of skin aging, protection and/or improving of a skin condition, and the prevention and/or treatment of skin imperfections are functional features which can be visualized, analyzed, measured and quantified using many techniques known by the specialist in cosmetic skin treatments. In some instances, the skin formulations described can be evaluated for their ability to up-regulate or down-regulate expression of one or more genes following one or more applications to the skin.

The methods of treating skin using the formulations described above can provide a skin rejuvenation effect and/or an improvement in the appearance of the skin, as measured via visual inspection, following step (1), as compared to the skin prior to step (1).

In some instances, the methods provide an effect of maintaining healthy skin; restoring damaged skin; enhancing the restoration of skin after cosmetic and dermatological procedures; enhancing wound healing; or combinations thereof.

In still other instances, the methods can treat, aleviate, or ameliorate one or more visible signs of aging of the skin, where the signs of aging can include, but are not limited to, fine lines, wrinkles, enlarged pores, roughness, dryness, loss of elasticity, loss of volume, atrophic skin, stretch marks, puffy eyes, dark (under eye) circles, and combinations thereof.

### BRIEF DESCRIPTION OF THE DRAWING

FIG. 1 is a bar graph showing the results of differential gene expression between sample treatments versus a placebo control group, as studied in Example 1.

FIG. 2A is a bar graph showing the results of differential Gene expression between sample treatments versus an untreated control group, as studied in Example 2.

FIG. 2B is a bar graph showing the results of differential Gene expression between sample treatments versus a placebo control group, as studied in Example 2.

FIG. 3A shows a table of a PCR Clinical Grading Scale for visual and tactile grading of skin.

FIG. 3B shows a table of a PCR Clinical Grading Scale for Vivascope assessment of collagen.

### DETAILED DESCRIPTION OF THE INVENTION

Cosmetic skin formulations and methods of using thereof are described herein.

### I. Definitions

Numerical ranges disclosed herein disclose individually each possible number in such range, as well as any sub-ranges and combinations of sub-ranges encompassed therein. For example, a carbon range (i.e., C₁-C₁₀) is intended to disclose individually every possible carbon value and/or sub-range encompassed within. For example, a carbon length range of C₁-C₁₀ discloses C₁, C₂, C₃, C₄, C₅, C₆, C₇, C₈, C₉, and C₁₀, as well as discloses sub-ranges encompassed within, such as C₂-C₉, C₃-C₈, C₁-C₅, etc. Similarly, an integer value range of 1-10 discloses the individual values of 1, 2, 3, 4, 5, 6, 7, 8, and 10, as well as sub-ranges encompassed within. Further, a concentration range or weight percent range, such as from 1% to 2% by weight of the formulation discloses, the individual values and fractions thereof, such as 1%, 1.1%, 1.2%, 1.32%, 1.48% etc., as well as sub-ranges encompassed within.

Use of the term “about” is intended to describe values either above or below the stated value, which the term “about” modifies, in a range of approx. +/−10%; in other instances the values may range in value either above or below the stated value in a range of approx. +/−5%. When the term “about” is used before a range of numbers (i.e., about 1-5) or before a series of numbers (i.e., about 1, 2, 3, 4, etc.) it is intended to modify both ends of the range of numbers or each of the numbers in the series, unless specified otherwise.

The term “skin” refers to the layer of tissue forming the natural outer covering of the body of a subject, typically a human or animal.

An “effective amount” refers to an amount of active agent(s), composition(s), and/or other component(s), as described herein, which can form part of a cosmetic skin formulation which, when applied as part of a desired skin treatment can achieve a desired result or effect, such as a skin rejuvenation effect, an improvement in the appearance of the skin, an effect of maintaining healthy skin, an effect of restoring damaged skin, an effect of enhancing the restoration of skin after cosmetic and/or dermatological procedures, an effect of enhancing wound healing, and/or an effect of treating, alleviating, or ameliorating one or more visible signs of aging of skin, when the skin is compared, such as by visual inspection, prior to and following treatment with a cosmetic skin formulation described. Other techniques for evaluating the effects of active agent(s), composition(s), and/or other component(s) on the skin are known, as described below.

The terms “topical” denotes when a formulation, as described herein, is applied to one or more areas of one or more surface layers of skin in a subject.

The term “pH balanced”, as used herein, refers to a formulation having pH from about 4 to about 7.

The terms “applying,” “administering,” or “administration,” as used herein, refer to topical application of the cosmetic skin formulation to areas of skin, such as by spreading, rubbing, or massaging.

The terms “aging of skin” or “signs of skin aging” include, but are not limited to, all outward visibly and tactilely perceptible manifestations as well as any other macro or micro effects due to skin aging. Such signs may be induced or caused by intrinsic factors (showing as chronological aged skin) and extrinsic factors (showing as environmental skin damage including but not limited photo-aged skin). These signs may result from processes which include, but are not limited to, the development of textural discontinuities such as wrinkles and coarse deep wrinkles, fine or skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including under eye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

“Cosmetically acceptable” refers to those compounds, materials, and formulations, which are, within the scope of sound medical judgment, suitable for use in contact with the tissues of human beings and animals without excessive toxicity, irritation, allergic response, or other problems or complications commensurate with a reasonable benefit/risk ratio. More specifically, cosmetically acceptable refers to a material, compound, or formulation which is suitable for use in contact with the skin, scalp, or hair. Cosmetically acceptable materials are known to those of ordinary skill in the art.

“Analog” and “Derivative” are used herein interchangeably and refer to a compound that possesses the same core as the parent compound, but differs from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be formed, at least theoretically, from the parent compound via chemical and/or physical processes.

“Electrophilic group” or “electrophilic moiety” are used interchangeably and refer to one or more functional groups or moieties that have an affinity for or attract electrons.

“Michael acceptor”, as used herein, is a species of electrophilic groups or moieties that participates in nucleophilic addition reactions. The Michael acceptor can be or can contain an α,β-unsaturated carbonyl-containing group or moiety, such as a ketone. Other Michael acceptors include pi-bonds, such as double or triple bonds conjugated to other pi-bond containing electron withdrawing groups, such as nitro groups, nitrile groups, and carboxylic acid groups.

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), or 20 or fewer carbon atoms, or 12 or fewer carbon atoms, or 8 or fewer carbon atoms. In some instances, the chain has 1-6 carbons. Likewise, cycloalkyls can have from 3-10 carbon atoms in their ring structure, or can have 5, 6 or 7 carbons in the ring structure. The ranges provided above are inclusive of all values between the minimum value and the maximum value.

The term “alkyl” includes both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths.

The alkyl groups may also contain one or more heteroatoms within the carbon backbone. Examples include oxygen, nitrogen, sulfur, and combinations thereof. In certain instances, the alkyl group contains between one and four heteroatoms.

“Alkenyl” and “Alkynyl”, as used herein, refer to unsaturated aliphatic groups containing one or more double or triple bonds analogous in length (e.g., C₂-C₃₀) and possible substitution to the alkyl groups described above.

“Aryl”, as used herein, refers to 5-, 6- and 7-membered aromatic rings. The ring may be a carbocyclic, heterocyclic, fused carbocyclic, fused heterocyclic, bicarbocyclic, or biheterocyclic ring system, optionally substituted as described above for alkyl. Broadly defined, “Ar”, as used herein, includes 5-, 6- and 7-membered single-ring aromatic groups that optionally includes from zero to four heteroatoms. Examples include, but are not limited to, benzene, pyrrole, furan, thiophene, imidazole, oxazole, thiazole, triazole, pyrazole, pyridine, pyrazine, pyridazine and pyrimidine. Those aryl groups having heteroatoms in the ring structure may also be referred to as “heteroaryl”, “aryl heterocycles”, or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN. The term “Ar” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic.

“Alkylaryl”, as used herein, refers to an alkyl group substituted with an aryl group (e.g., an aromatic or hetero aromatic group).

“Heterocycle” or “heterocyclic”, as used herein, refers to a cyclic radical attached via a ring carbon or nitrogen of a monocyclic or bicyclic ring containing 3-10 ring atoms, or from 5-6 ring atoms, containing carbon and one to four heteroatoms each selected from non-peroxide oxygen, sulfur, and N(Y) wherein Y is absent or is H, O, (C₁₋₄) alkyl, phenyl or benzyl, and optionally containing one or more double or triple bonds, and optionally substituted with one or more substituents. The term “heterocycle” also encompasses substituted and unsubstituted heteroaryl rings. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Heteroaryl”, as used herein, refers to a monocyclic aromatic ring containing five or six ring atoms containing carbon and 1, 2, 3, or 4 heteroatoms each selected from non-peroxide oxygen, sulfur, and N(Y) where Y is absent or is H, O, (C₁-C₈) alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl include, but are not limited to, furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

“Halogen”, as used herein, refers to fluorine, chlorine, bromine, or iodine.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, such from 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms, such as nitrogen, may have hydrogen substituents and/or any permissible substituents of organic compounds described herein that satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

“Polymer”, as used herein, refers to a molecule containing more than 10 monomer units.

“Water-soluble”, as used herein, generally means at least 50, 75, 100, 125, 150, 200, 225, or 250 g is soluble in 1 L of water at 25° C.

### II. Cosmetic Skin Formulations

Cosmetic skin formulations are described herein.

### A. Exemplary Cosmetic Skin Formulations

In a non-limiting instance, the cosmetic skin formulation includes:
(i′) a first composition including glycerin and Lavandula angustifolia extract;
(ii′) at least one of:
   (a) a second composition including Vibrio alginolyticus ferment filtrate;
   (b) a third composition including acetyl tyrosine, proline, and adenosine triphosphate;
   (c) cetylhydroxyproline palmitamide;
   (d) N-Succinyl-S-Famesyl-L-Cysteine;
(iii′) one or more dermatologically acceptable carriers; and
(iv′) optionally one or more dermatologically acceptable excipients.

In another non-limiting instance, the cosmetic skin formulation includes:
(i″) a first composition including glycerin and Lavandula angustifolia extract;
(ii″) N-Succinyl-S-Farnesyl-L-Cysteine; and
(iii″) optionally at least one of:
   (a) a second composition including Vibrio alginolyticus ferment filtrate;
   (b) a third composition including acetyl tyrosine, proline, and adenosine triphosphate;
   (c) cetylhydroxyproline palmitamide;
(iv″) one or more dermatologically acceptable carriers; and
(v″) optionally one or more dermatologically acceptable excipients.

In yet another non-limiting instance, the cosmetic skin formulation includes:
(i′″) a first composition including glycerin and Lavandula angustifolia extract;
(ii′″) cetylhydroxyproline palmitamide; and
(iii′″) optionally at least one of:
   (a) a second composition including Vibrio alginolyticus ferment filtrate;
   (b) a third composition including acetyl tyrosine, proline, and adenosine triphosphate;
   (c) N-Succinyl-S-Farnesyl-L-Cysteine;
(iv′″) one or more dermatologically acceptable carriers; and
(v′″) optionally one or more dermatologically acceptable excipients.

In still another non-limiting instance, the cosmetic skin formulation includes:
(i″″) a first composition including glycerin and Lavandula angustifolia extract;
(ii″″) a second composition including Vibrio alginolyticus ferment filtrate; and
(iii″″) optionally at least one of:
   (a) a third composition including acetyl tyrosine, proline, and adenosine triphosphate;
   (b) N-Succinyl-S-Farnesyl-L-Cysteine;
   (c) cetylhydroxyproline palmitamide;
(iv″″) one or more dermatologically acceptable carriers; and
   (v″″) optionally one or more dermatologically acceptable excipients.

In another non-limiting instance, the cosmetic skin formulation includes:
(i′″″) a first composition including acetyl tyrosine, proline, and adenosine triphosphate;
(ii′″″) at least one of:
   (a) a second composition including Vibrio alginolyticus ferment filtrate;
   (b) a third composition including glycerin and Lavandula angustifolia extract;
   (c) cetylhydroxyproline palmitamide; and/or
(d) N-Succinyl-S-Famesyl-L-Cysteine;
(iii′″″) one or more dermatologically acceptable carriers; and
(iv′″″) optionally one or more dermatologically acceptable excipients.

In some instances, the cosmetic skin formulation above, which includes at least one component of components (a)-(d), includes at least two of components (a)-(d); or at least three of components (a)-(d). In still other instances, the skin formulation includes each of components (a)-(d).

In some instances, the cosmetic skin formulation above, which includes at least one component of components (a)-(c), includes at least two of components (a)-(e), In still other instances, the skin formulation includes each of components (a)-(c).

The cosmetic skin formulations disclosed herein are applied to the skin of a subject, such as a mammal (typically a human). The one or more dermatologically acceptable carriers and/or excipients are considered safe for application to human skin, as well as to hair, and may be administered to an individual's skin without causing undesirable biological side effects, such as burning, stinging, itching, tingling, irritation, and/or redness, or similar adverse reactions at the site of application.

In still another instance, the cosmetic skin formulation is a scalp formulation that includes:
(1′) at least one active agent of Formula I (as described below); and a
(2′) a first composition comprising glycerin and Lavandula angustifolia extract;
(3′) optionally one or more dermatologically acceptable excipients; and
(4′) one or more dermatologically acceptable carriers.

The one or more dermatologically acceptable excipients and one or more dermatologically acceptable carriers can be selected from the lists described elsewhere herein.

In some instances, the active agent in the scalp formulation is bis-aminopropyl diglycol dimaleate, which can be represented by the following chemical structure:

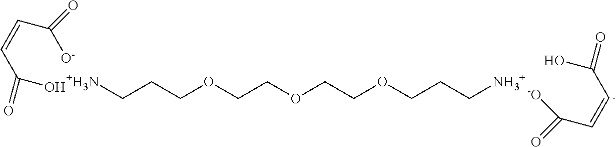

In some instances, the scalp formulation is in the form of a serum.

The cosmetic skin formulations disclosed herein can have any pH suitable for application to skin. In some instances, the cosmetic skin formulations have a pH in a range of about 4 to about 7, or at a pH of 4.4 to 6, as well as individual pH values or sub-ranges contained within the aforementioned ranges, or at a pH of 5.5. In certain instances, the skin formulations are adjusted to provide a pH balanced skin formulation.

a. Active Agent(s), Composition(s), and Other Optional Component(s) of Cosmetic Skin Formulations

The cosmetic skin formulations disclosed herein typically include a combination of agent(s), composition(s), and other optional component(s). It is understood that the skin formulation is a blend of these agent(s), composition(s), and other optional component(s) and typically forms a homogeneous composition thereof.

i. Agents Defined by Formula I

In some instances, at least one active agent of Formula I is included in the skin formulations which contains a reactive moiety which is capable of reacting with and forming bonds with a nucleophile, such as a thiol. Such reactive moieties, upon reaction with thiol groups, are able to form bonds that are stable, for example, hydrolytically stable. “Stable”, as used in reference to the bonds formed between thiol groups where the bonds remain intact for at least one week, two weeks, three weeks, four weeks, one month, or two months or longer when exposed to water at a pH of about 4 to about 6, or at a pH of 4.4 to 6, or at a pH of 5.5 at a temperature from about 5° C. to about 100° C., from about 20° C. to about 75° C., or from about 20° C. to about 50° C., from about 25° C. to about 40° C., or from about 25° C. to about 30° C., or at a temperature of about 25° C. In some instances, the temperature is about 25° C. The reaction between the reactive moieties and thiol typically occurs around room temperature, for example, from about 15° C. to about 35° C., or from about 20° C. to about 30° C., or from about 22° C. to about 27° C.

As previously noted, an active agent according to Formula I, can have a structure as shown below:

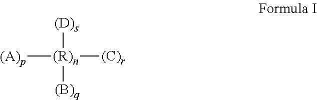

wherein A, B, C, and D are reactive moieties containing one or more charges,
R is a linker that contains two or more charges, wherein the charges are opposite to the charges on the reactive moieties, wherein n=1-100, preferably n=1-10, more preferably n=1,
each occurrence of p, q, r, and s is independently an integer from 0 to 25, preferably from 0 to 10, more preferably from 0 to 2. The sum of p+q+r+s is equal to or greater than 2.

The reactive moieties may be present on any atom of the linker. In some instances, the reactive moieties are the same. In some instances, one or more of the reactive moieties is different.

In some instances, the reactive moieties are negatively charged and the linker has positively charged moieties. In other instances, the reactive moieties are positively charged and the linker or spacer has negatively charged moieties. Generally, the sum of the charges on the active agent of Formula I is zero, though stoichiometric imbalances may exist.

The reactive moieties on the active agents are typically linked via a linker R. The linker R, as used herein, refers to one or more polyfunctional, e.g. bifunctional molecules, trifunctional molecules, tetrafunctional molecules, etc., which can be used to ionically bind the two or more reactive moieties and which do not interfere with the reactive properties of the reactive moeities. The reactive moieties may be attached to any part of linker R.

### 1. Linker R

In certain instances, for Formula I, n=1, and the linker R is not a polymer. In some instances, the linker R can be a single atom, such as a metal ion (i.e., Ca²⁺) or multiple groups of atoms, such as an alkyl, alkoxy, or alkenyl chain. Suitable linkers include but are not limited to oxygen, sulfur, carbon, boron, nitrogen, alkoxy, alkyl, alkenyl, cycloalkyl, cycloalkenyl, aryl, heterocycloalkyl, heteroaryl, ether, amine, and a polymer.

The linker R can be optionally independently substituted with one or more substituents including hydrogen, halogen, cyano, alkoxy, alkyl, alkenyl, cycloalkyl, cycloalkenyl, aryl, heterocycloalkyl, heteroaryl, amine, hydroxy, formyl, acyl, carboxylic acid (—COOH), —C(O)R¹, —C(O)OR¹, carboxylate (—COO—), primary amide (e.g., —CONH₂), secondary amide (e.g., —CONHR₁), —C(O)NR¹R², —NR¹R², —NR¹S(O)₂R², —NR¹C(O)R², —S(O)₂R², —SR¹, and —S(O)₂NR¹R², sulfinyl group (e.g., —SOR¹), and sulfonyl group (e.g., —SOOR¹); wherein R¹ and R² may each independently be hydrogen, alkyl, alkenyl, alkynyl, cycloalkyl, aryl, heterocycloalkyl and heteroaryl; wherein each of R¹ and R² is optionally independently substituted with one or more substituents selected from the group consisting of halogen, hydroxyl, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxyl, cycloalkyl optionally substituted with hydroxyl, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl.

In some instances, the linker R may be an alkoxy, ether, alkyl, alkenyl, cycloalkyl, cycloalkenyl, aryl, heterocycloalkyl, heteroaryl, amine based linker. In yet other instances, the linker is a polyfunctional molecule which is an alkoxy or alkenyl molecule and the linker is substituted with one or more substituents selected from the group consisting of hydrogen, halogen, cyano, alkenyl, cycloalkyl, cycloalkenyl, amine, hydroxy, formyl, acyl, primary amide, secondary amide, —C(O)NR¹R², —NR¹R², —NR¹S(O)₂R², —NR¹C(O)R², —S(O)₂R², —SR¹, and
—S(O)₂NR¹R², sulfinyl group, and sulfonyl group; wherein R¹ and R² may each independently be hydrogen, alkyl, alkenyl, alkynyl, cycloalkyl, aryl, heterocycloalkyl, or heteroaryl; where each of R¹ and R² is optionally independently substituted with one or more substituents selected from the group consisting of halogen, hydroxyl, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxyl, cycloalkyl optionally substituted with hydroxyl, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl, and dialkylaminocarbonyl; or the linker may be a polyfunctional molecule which is an alkyl molecule, which is substituted or unsubstituted, and when substituted the alkyl molecule substituents are selected from the group consisting of halogen, hydroxyl, thiocarbonyl alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, an aromatic, and a heteroaromatic group.

The reaction between the reactive moiety and the thiol groups may be initiated at room temperature and pressure when the reactive moiety contacts a thiol group, such as in skin. In some other instances, the reaction may include an initiator, such as heat, catalyst, basic conditions, and/or a free radical initiator. The rate of reaction between the reactive moiety and the thiol may be increased by changes in temperature, pH, and/or addition of one or more excipients, such as a catalyst; however, this is generally not required.

In some instances, the reactive moieties are capable of undergoing a conjugate addition reaction. The reactive moieties can independently be or contain a Michael acceptor, a succinimidyl-containing group, a maleimido-containing group, azlactone, a benzoxazinone derivative, vinyl sulfone, vinyl sulfoximine, vinyl sulfonate, vinyl phosphonate, benzoxazinone, isocyanate, epoxide, an electrophilic moiety containing a leaving group, an electrophilic thiol acceptor, acrylic or acrylate group, a methacrylic or methacrylate group, a styrene group, an acryl amide group, a methacryl amide group, a maleate group, a fumarate group, an itaconate group, a vinyl ether group, an allyl ether group, an allyl ester group, a vinyl ester group, a sulfonate group, a phosphonate group, a sulfoxide group, a sulfonamide group, a sulfinimide group, a sulfinamide group, a sulfonimidate group, or a sulfonimidamide group.

In certain instances, each of reactive moieties A, B, C, and/or D when present independently contains a moiety selected from the group consisting of a vinyl sulfone, an acrylate group, a methacrylate group, a styrene group, an acryl amide group, a methacryl amide group, a maleate group, a fumarate group, and an itaconate group. Further, in preferred instances, n=1 and the linker R is not a polymer. Optionally, all of the reactive moieties are the same. For example, in some instances all of the reactive moieties are maleate groups.

In some instances, the active agent according to Formula I can be represented by one of the following chemical structures:

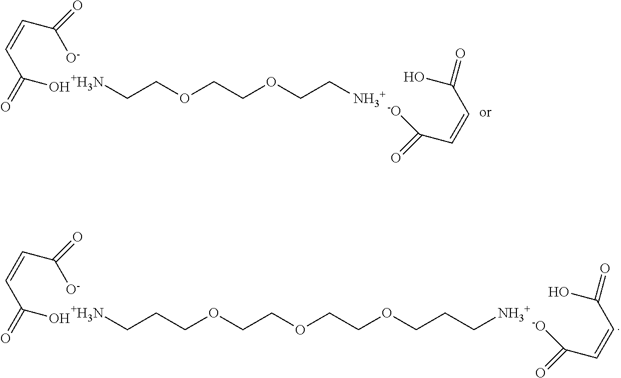

In some particular instances, the active agent according to Formula I is bis-aminopropyl diglycol dimaleate, which can be represented as shown below:

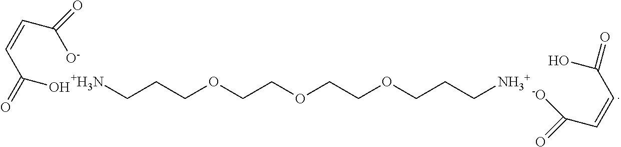

Active agents according to Formula I are further described in U.S. Patent Publication No. 2015/0034117 A1 to Pressly, et al., which is incorporated herein by reference with respect to its disclosure of active agents.

The active agent is typically present in an amount ranging from about 0.001 wt % to about 25 wt % of the total weight of the cosmetic skin formulation, from about from about 0.001 wt % to about 15 wt % of the total weight of the cosmetic skin formulation, from about 0.001 wt % to about 10 wt % of the total weight of the cosmetic skin formulation, or from about 0.001 wt % to about 5 wt % of the total weight of the cosmetic skin formulation, as well as sub-ranges or individual concentration values disclosed within the aforementioned ranges. Typically, the active agent may be present in an amount ranging from about 0.001 to about 5 wt %, about 0.001 wt % to about 2.5 wt %, or about 0.001 wt % to about 1 wt % of the total weight of the cosmetic skin formulation, as well as sub-ranges or individual concentration values disclosed within the aforementioned range. The “total weight of the formulation” generally refers to the sum of weights of all active agents, compositions, other components, carriers, excipients, and/or additives, as may be present, in a given cosmetic skin formulation.

The active agent is stable in the skin formulation for a period of at least 2, 3, 4, 5, 6, 8, 9, 10, 11, or 12 months or longer at pH of about 4 to about 7, or at a pH of 4.4 to 7, and individual pH values or sub-ranges contained within the aforementioned ranges, or at a pH of 5.5, and a temperature of about 25-30° C., such as about 25° C. “Stable” as used herein with respect to shelf-life means that at least 40, 45, 50, 55, 60, 65, 70, 75, 80, 85, 90, or 95% of the compound is unchanged over the specified time period.

In one instance, a cosmetic skin formulation is a moisturizer formulation which includes a compound of Formula I, such as bis-aminopropyl diglycol dimaleate. The cosmetic skin formulation also includes: a first composition including glycerin and Lavandula angustifolia extract; a second composition including Vibrio alginolyticus ferment filtrate; cetylhydroxyproline palmitamide; one or more dermatologically acceptable carriers; and one or more dermatologically acceptable excipients, as described above.

In some instances, the cosmetic skin formulation is a moisturizer formulation which includes: a compound of Formula I, such as bis-aminopropyl diglycol dimaleate, water, sodium polyglutamate, hydroxyacetophenone, sclerotium gum, glycerin, 1,2-hexanediol, caprylyl glycol, cetearyl alcohol, cetearyl glucoside, glucose, polyglyceryl-4 oleate, polyglyceryl-3 polyricinoleate, glyceryl oleate citrate, C₁₀-C₁₈ triglycerides, C₁₃-C₁₅ alkane (hemisqualane), squalane, capryloyl glycerin/sebacic acid copolymer, VP/hexadecene copolymer, diisostearyl malate, caprylic/capric triglyceride, salicornia herbacea extract, cetylhydroxyproline palmitamide, hydroxyethyl acrylate/sodium acryloyldimethyl taurate copolymer, sorbitan isostearate, polyacrylate crosspolymer-6, t-butyl alcohol, hydrogenated polydecene, glyceryl stearate, ceramide Np, hydrogenated lecithin, cholesterol, stearic acid, ceramide Ap, phytosphingosine, tetraacetylphytosphingosine, xanthan gum, ceramide Eop, glycosphingolipids, beta-glucan, sodium benzoate, pentylene glycol, sodium acetylated hyaluronate, sodium hyaluronate, sodium hyaluronate crosspolymer, hydrolyzed sodium hyaluronate, ethylhexylglycerin, saccharide isomerate, sodium citrate, citric acid, pentylene glycol, fructose, urea, sodium hydroxide, maltose, sodium PCA, sodium chloride, sodium lactate, trehalose, allantoin, Lavandula angustifolia flower/leaf/stem extract, butylene glycol, Vibrio alginolyticus ferment filtrate, and sodium dilauramidoglutamide lysine.

In another instance, a cosmetic skin formulation is a scalp formulation which includes a compound of Formula I, such as bis-aminopropyl diglycol dimaleate. The scalp formulation also includes: a first composition including glycerin and Lavandula angustifolia extract; one or more dermatologically acceptable carriers, as described above; and optionally one or more dermatologically acceptable excipients, as described above.

In some instances, the cosmetic skin formulation is a scalp formulation, which includes: water, propanediol, glycerin, chlorphenesin, Glycine soja (soybean) seed extract, sodium acrylates copolymer, lecithin, C₉-C₁₂ alkane, coco-caprylate/caprate, Oryza sativa (rice) bran oil, octyldodecanol, Leptospermum scoparium branch/leaf oil, Magnolia officinalis bark extract, Piper nigrum (pepper) seed extract, menthoxypropanediol, hydrogenated polydecene, 1,2-hexanediol, ceramide NP, cetearyl alcohol, glyceryl stearate, ceramide AP, cholesterol, hydrogenated lecithin, phytosphingosine, stearic acid, tetraacetylphytosphingosine, xanthan gum, ceramide EOP, glycosphingolipids, pogostemon cablin leaf/stem extract, phenethyl alcohol, Lavandula angustifolia (lavender) flower/leaf/stem extract, Tasmannia lanceolata leaf extract, pentylene glycol, sodium acetylated hyaluronate, sodium hyaluronate, sodium hyaluronate crosspolymer, hydrolyzed sodium hyaluronate, ethylhexylglycerin, bis-aminopropyl diglycol dimaleate, phenoxyethanol, sodium benzoate, citric acid, and arginine.

In some instances, the scalp formulation contains the compound of Formula I, such as bis-aminopropyl diglycol dimaleate, and the first composition including glycerin and Lavandula angustifolia extract in concentrations effective to increase barrier function and reduce transepidermal water loss (TEWL) of the scalp, as compared to the scalp prior to application of the scalp formulation thereon.

b. Composition(s) and Other Component(s)

The cosmetic skin formulations can include combinations of different compositions, such as:
a composition including glycerin and Lavandula angustifolia extract;
a composition including Vibrio alginolyticus ferment filtrate; and/or
a composition including acetyl tyrosine, proline, and adenosine triphosphate.

In some instances, the composition including Vibrio alginolyticus ferment filtrate further includes butylene glycol. In some instances, the composition including acetyl tyrosine, proline, and adenosine triphosphate further includes butylene glycol and/or hydrolyzed vegetable protein.

The cosmetic skin formulations can also include other agents/components, such as:
cetylhydroxyproline palmitamide;
N-Succinyl-S-Farnesyl-L-Cysteine;
neuromide;
hydroxysomes calcium.

As noted above, the cosmetic skin formulations may also include an active agent of Formula I, such as bis-aminopropyl diglycol dimaleate.

Each of the above compositions and other agents/components can independently be present in an amount ranging from about 0.001 wt % to about 15 wt % of the total weight of the cosmetic skin formulation, from about 0.001 wt % to about 10 wt % of the total weight of the cosmetic skin formulation, from about 0.001 wt % to about 5 wt % of the total weight of the cosmetic skin formulation, from about 0.001 wt % to about 2.5 wt % of the total weight of the cosmetic skin formulation, or from about 0.001 wt % to about 1 wt % of the total weight of the cosmetic skin formulation, as well as sub-ranges or individual concentration values disclosed within the aforementioned ranges.

In some other instances, each of the above compositions and other agents/components can independently be present in an amount based on the relative cytotoxicity of the composition, agent, or component. In some instances, the dose of EXO-T™ composition in the skin formulation does not exceed about 10,000 μg/mL. In some instances, the dose of WASTAR™ Lavender Composition in the skin formulation does not exceed about 10,000 μg/mL. In some instances, the dose of UNIREPAIR® T-43 Composition in the skin formulation does not exceed about 1,000 μg/mL. In some instances, the dose of SFC component in a skin formulation does not exceed about 0.1 μg/mL.

Without limitation, cytotoxicity may be evaluated using an MTS-cytoxicity assay with a reagent, such as cell-titer One-Solution MTS Assay, and equipment, such as Envision plate reader (Perkin-Elmer). In an exemplary procedure:
a) after incubation of skin cells with the test material (i.e., composition, agent, or component) being evaluated in plate wells at various concentrations, remove treatment media from plate wells;
b) add 0.1 mL of 10% FBS-DMEM-Phenol red free-MTS-media per well;
c) incubate plate well at 37° C. and 5% CO₂ for 30 min;
d) read absorbance at 490 nm using a plate reader;
e) calculate percent (%) viability per treatment concentration according to equation below:

$$\% viability = \frac{{Treatment}_{OD}}{Untreated or Vehicle {control}_{OD}} \times 100$$

f) determine the highest non-toxic concentration (>80% cell viability) after 24 hours treatments per test material.

It is understood that the sum of percentages of all compositions and other components, as may be present, the one or more dermatologically acceptable carriers, and the one or more optional dermatologically acceptable excipients will total 100%. The aforementioned may be present in any given skin formulation at any suitable concentration and/or ratio between any two or more components, compositions, carriers, and excipients, as may be present.

Each of the compositions and other components are stable in the skin formulation for a period of at least 2, 3, 4, 5, 6, 8, 9, 10, 11, or 12 months or longer at pH of about 4 to about 6, or at a pH of 4.4 to 6, or at a pH of 5.5, and a temperature of about 25-30° C., or about 25° C. “Stable” as used herein with respect to shelf-life means that at least 40, 45, 50, 55, 60, 65, 70, 75, 80, 85, 90, or 95% of the compound is unchanged over the specified time period.

The different compositions and other components named above may be obtained from commercial sources, such as detailed below.

### 1. WASTAR™ Lavender Composition

The WASTAR™ lavender composition includes glycerin and Lavandula angustifolia extract in water. The WASTAR™ lavender composition is commercially produced as WASTAR™ Lavender by Lucas Meyer Cosmetics. WASTAR™ Lavender is an anti-pollution, anti-stress, and protective agent which is described as an upcycled lavender extract formed from by-products remaining after naturals essential oil production WASTAR™ Lavender is an active ingredient which can provide protection to skin against pollution.

Regarding the Lavandula angustifolia extract, the term “extract” may be an extract obtained from the flowers, leaves, fruits, seeds, roots and/or stems of lavender plant. Lavender belongs to the genus Lavandula, the family Lamiaceae, the order Lamiales, the class Dicotyledoneae. In certain instances, the Lavandula angustifolia extract may be an extract obtained from the flowers, leaves, and/or stems of lavender, and the extract has moderate to high water-solubility (where moderate solubility is about 10-1,000 mg/L and high solubility is more than 1,000 mg/L total dissolution of the extract in water). In some other instances, the Lavandula angustifolia extract is an oil obtained from the seeds, flowers, leaves, and/or stems, where the extract is water-insoluble (having less than about 10 mg/L total dissolution of the extract in water). In some instances, the Lavandula angustifolia extract is not an oil or does not include any oil obtained from lavender

### 2. EXO-T™ Composition

The EXO-T™ composition includes Vibrio alginolyticus ferment filtrate; with water and butylene glycol as excipients. The composition is commercially produced as EXO-T™ by Lucas Meyer Cosmetics. EXO-T™ is an exopolysaccharide from Kopara (microorganisms mat) living in unique ecosystem in the rims of French Polynesian atolls.

EXO-T™ can improve skin smoothness leading to an even skin complexion and also help to reduce the appearance of stretch marks and age spots.

### 3. UNIREPAIR® T-43 Composition

The UNIREPAIR® T-43 composition includes acetyl tyrosine, proline, and adenosine triphosphate; with water, butylene glycol, and hydrolyzed vegetable protein as excipients. The composition is commercially produced as UNIREPAIR® T-43 by Givaudan Active Beauty. UNIREPAIR® T-43 is an amino acid rich complex which can be used to reduce skin erythema. UNIREPAIR® T-43 is also said to act as a regenerating/repairing/revitalizing, anti-oxidant, anti-redness, UV/sun protection, and SPF booster.

### 4. CeramideBio Component

The CeramideBio component is formed of cetylhydroxyproline palmitamide. The component is commercially produced as CeramideBio by Symrise. CeramideBio is a pseudoceramide resembling natural Ceramide 2 in structure and activity. Ceramides constitute one of the 3 key lipid components of the skin's permeability barrier (in the outer horny layer). CeramideBio is a preservative free wax which can be used to maintain intact skin and hair, as well as to support the repair of damaged skin and hair.

### 5. SFC Component

The SFC component is formed of N-Succinyl-S-farnesyl-L-cysteine (SFC). This component is commercially produced by Signum Bioscience. SFC is an anti-inflammatory small molecule and is the first farnesyl-cysteine IPC shown to clinically improve appearance and signs of aging, while also having the potential to ameliorate inflammatory skin conditions.

In addition to the above-named components, at least one of which is present in the cosmetic skin formulation, the skin formulation optionally includes one or more of the following additional components.

### 6. Neuromide Component

An optional component is Neuromide® (Dr. Raymond Laboratories, Inc.). Neuromide® is included in skin care formulations, and contains N-Palmitoyl serinol.

### 7. Hydroxysomes® Calcium Component

An optional component is Hydroxysomes® calcium (Laboratory Skin Care, Inc.). Hydroxysomes® calcium is a micro-carrier dermal delivery platform containing nanoporous calcium phosphate particles (average particle size 2-4 mm).

### 8. HydroSal® Moisturizer Component

An optional component is Natural HydroSal® Moisturizer (Salvona, LLC). Natural HydroSal® Moisturizer includes water (aqua), polyglyceryl-4 caprate, Helianthus annuus (Sunflower) seed oil, ceramide NP, ceramide AP, ceramide NG, phytosphingosine, phytosterols, lysolecithin, sclerotium gum, xanthan gum, pullulan, acacia senegal gum, bentonite, tragacanth gum, pentylene glycol, caprylyl glycol, propanediol, and ethylhexylglycerin.

### 9. Hymagic™-4D Component

An optional component includes a formulation of sodium hyaluronate crosspolymer, sodium hyaluronate, sodium acetylated hyaluronate, hydrolyzed sodium hyaluronate, pentylene glycol, ethylhexylglycerin, and water (aqua). This optional component is commercially produced as Hymagic™-4D by Bloom Biotechnology Corporation, Ltd.

### 10. MINHIHA™ Component

An optional component is formed of hydrolyzed sodium hyaluronate. The optional component is commercially available as MINIHA™ by Bloom Biotechnology Corporation, Ltd.

### 11. PRORENEW COMPLEX CLR™

### Component

An optional component is formed of Lactococcus ferment lysate. This

optional component is commercially available as PRORENEW COMPLEX CLR™ by CLR Chemisches Laboratorium Dr. Kurt Richter GmbH.

### 12. DRAGOSANTOL®100 Component

An optional component includes bisabolol, a monocyclic, unsaturated sesquiterpene alcohol. This component is commercially available as DRAGOSANTOL® 100 by Symrise.

Regarding the aforementioned optional components, as discussed above, each of the optional components, when present, can be present in an amount ranging from about 0.001 wt % to about 15 wt % of the total weight of the cosmetic skin formulation, from about 0.001 wt % to about 10 wt % of the total weight of the cosmetic skin formulation, from about 0.001 wt % to about 5 wt % of the total weight of the cosmetic skin formulation, from about 0.001 wt % to about 2.5 wt % of the total weight of the cosmetic skin formulation, or from about 0.001 wt % to about 1 wt % of the total weight of the cosmetic skin formulation, as well as sub-ranges or individual concentration values disclosed within the aforementioned ranges.

Each of the optional components are stable in the skin formulation for a period of at least 2, 3, 4, 5, 6, 8, 9, 10, 11, or 12 months or longer at pH of about 4 to about 6, or at a pH of 4.4 to 6, or at a pH of 5.5, and a temperature of about 25-30° C., or about 25° C. and 1 atm. “Stable” as used herein with respect to shelf-life means that at least 40, 45, 50, 55, 60, 65, 70, 75, 80, 85, 90, or 95% of the compound is unchanged over the specified time period.

c. Carriers, Optional Excipients, and Optional Additives

The cosmetic skin formulations described herein typically include one or more dermatologically acceptable carriers. Such carriers can be water, dermatologically acceptable organic solvents, or a combination thereof.

The total weight of the carriers, excipients, and/or additives refers to the sum of the weights of each component in the formulation in the particular category (e.g. carrier, excipient or additive). For example, two or more dermatologically acceptable carriers are included in the skin formulation, the total weight of the dermatologically acceptable carriers refers to the sum of the weight for each dermatologically acceptable carrier in the formulation. To obtain the weight percentage for the carriers, excipients, and/or additives in the formulation, the total weight for the particular category (i.e. carrier, excipient or additive) is divided by the total weight of all of the components in the skin formulation and multiplied by 100.

The total weight of the one or more dermatologically acceptable carriers can be in an amount ranging from about 0.01 wt % to about 99 wt % or 0.01 wt % to about 95 wt % of the total weight of skin formulation, as well as sub-ranges or individual concentration values contained within the aforementioned ranges. In some instances, the total weight of the one or more dermatologically acceptable carriers ranges from about 0.1 wt % to about 90 wt %, about 0.1 wt % to about 80 wt %, about 0.1 wt % to about 70 wt %, about 0.1 wt % to about 60 wt %, about 0.1 wt % to about 50 wt %, about 0.1 wt % to about 40 wt %, about 0.1 wt % to about 30 wt %, about 0.1 wt % to about 20 wt %, or about 0.1 wt % to about 10 wt % of the total weight of the skin formulation, as well as sub-ranges or individual concentration values contained within the aforementioned ranges. In some instances, the cosmetic skin formulations can be free or substantially free of any one or more of the aforementioned dermatologically acceptable carriers, where “substantially free” refers to the formulation including less than about 3%, 2%, 1%, 0.9%, 0.8%, 0.7%, 0.6%, 0.5%, 0.4%, 0.3%, 0.2%, or 0.1% by weight in the total weight of the skin formulation. It is understood that for the aforementioned ranges that the values are indicative of the total sum of the concentration(s) of any single or combination of dermatologically acceptable carriers, as may be present.

In some instances, the cosmetic skin formulation includes one or more dermatologically acceptable excipients. For example, the dermatologically acceptable excipient can be an abrasive, an acidulent, an adhesion promoter, an antioxidant, an antiseptic, an astringent, a barrier agent, a bio-adhesive agent, a botanical agent, a botanical extract, a buffer agent (such as lactic acid, sodium lactate, citric acid, trisodium citrate, sodium hydroxide), a bulking agent, a calming agent, a chelating agent (such as EDTA, tetrasodium glutamate diacetate), a cleansing agent, an emulsifier agent, a colorant, a conditioning agent, a cooling agent, a deodorant agent, a depilatory agent, a detergent, a disinfectant, a dispersant (such as hydroxyethyl acrylate/sodium acryloyldimethyl tauronate copolymer), an emollient, an emulsifier, an emulsion stabilizer, an essential oil, an exfoliant, a fat soluble agent, a fiber, a film former, a fixative, a foam booster, a foam stabilizer, a foaming agent, a fragrance, a free radical scavenger, a fungicide, a gellant, a glosser, a bleaching agent, a conditioning agent, a humectant, a lathering agent, a lubricant, a make-up agent (such as to provide a color, tint, gloss, or matte to the formulation and/or the skin to which it is applied), a moisture barrier agent, a moisturizing agent, a neutralizer, an odor-masking agent, an oil, an oil absorbent agent, an ointment base, an opacifier, an organosilicone, an oxidant, an oxygen carrier, a pearlant agent, a perfume, a perfume solvent, a perfume stabilizer, a peroxide stabilizer, a photo-sensitizer agent, a pigment, a pigmenting agent, a pearlescent aid, a plant extract, a plant derivative, a plant tissue extract, a plant root extract, a plant seed extract, a plant oil, a plasticizer, a polymer, a polymer film former, a preservative agent, a propellant, a reducing agent, a re-fatting agent, a resin, a scalp agent, a scrub agent, a sequestrant, a silicone agent, a skin clarifier, a skin cleanser, a skin lipid, a skin lightening agent, a skin bleaching agent, a solubilizer, a suspending agent, a sun protection agent, a soothing agent (such as bisabolol), a spreading agent, a stabilizer, a sunless tanning agent, a sunscreen, a UVA sunscreen, a UVB sunscreen, a broad-band sunscreen, a surfactant, an amphoteric surfactant, an anionic surfactant, a cationic surfactant, a non-ionic surfactant, a silicone surfactant, an accelerator, a thickening agent, a thixotrope, a tightening agent, a toner, a tonic agent, a vegetable oil, a volatile agent, a viscosity stabilizer, a vitamin, a water proofing agent, a wax, a wetting agent, a whitening agent, or a combination thereof.

The total weight of dermatologically acceptable excipient(s) can be in a range from about 0.001 wt % to about 99 wt %, 0.001 wt % to about 95 wt %, or 0.001 wt % to about 90 wt % of the total weight of the skin formulation, as well as individual concentration values or sub-ranges contained within the aforementioned ranges. In some instances, the total weight of dermatologically acceptable excipient(s) can range from about 0.1 wt % to about 95 wt %, about 0.1 wt % to about 90 wt %, about 0.1 wt % to about 80 wt %, about 0.1 wt % to about 70 wt %, about 0.1 wt % to about 60 wt %, about 0.1 wt % to about 50 wt %, about 0.1 wt % to about 40 wt %, about 0.1 wt % to about 30 wt %, about 0.1 wt % to about 20 wt %, or about 0.1 wt % to about 10 wt % of the total weight of the skin formulation, as well as sub-ranges or individual concentration values contained within the aforementioned ranges. In some instances, the cosmetic skin formulations can be free or substantially free of any of the one or more of the aforementioned dermatologically acceptable excipients, where “substantially free” refers to the formulation including less than about 3%, 2%, 1%, 0.9%, 0.8%, 0.7%, 0.6%, 0.5%, 0.4%, 0.3%, 0.2%, or 0.1% by weight of dermatologically acceptable excipient(s) in the total weight of the skin formulation. It is understood that for the aforementioned ranges that the values are indicative of the total sum of the concentration(s) of any single or combination of dermatologically acceptable excipients, as may be present.

In some instances, the cosmetic skin formulations can further include one or more additives. For example, the additive can be an agent modulating cell differentiation, an agent modulating cell proliferation, an agent stimulating synthesis of dermal or epidermal macromolecules, an agent preventing degradation of dermal or epidermal macromolecules, an agent acting on microcirculation, an antimicrobial sequestering agent, an analgesic, an anesthetic, an anti-acne agent, an anti-aging agent, an anti-wrinkle agent, an anti-atrophy agent, an anti-bacterial agent, an anti-scar agent, an anti-seborrheic agent, an anti-cracking agent, an anti-cellulite agent, an anti-stretch mark agent, an anti-dandruff agent, an anti-fungal agent, an antihistamine agent, an anti-inflammatory agent, an anti-irritant agent, an anti-microbial agent, an antibiotic agent, an antiviral agent, an anti-skin cancer agent, an anti-eczema agent, an anti-psoriasis agent, an antiperspirant agent, an anti-pruriginous agent, an anti-pruritic agent, a circulatory stimulant agent, a collagen stimulating agent, an elastin stimulating agent, an extracellular matrix stimulating agent, an enzyme, an enzymatic inhibitor, an enzyme-inducing agent, a hormone, a hormone-like agent, a hyaluronic acid stimulating agent, a keratolytic agent, a muco-adhesive agent, a natural moisturizing agent, a lipolytic agent, a peptide agent (such as palmitoyl tripeptide-1, palmitoyl tetrapeptide-7), a protein agent, a rosacea inhibitory agent, a scar prevention agent, a skin barrier agent, a skin barrier restoration agent, a skin calming agent, a skin smoothing agent, a skin calming agent, a skin soothing agent (such as beta-glucan), a skin penetration enhancing agent, a skin penetration retarding agent, a skin conditioning agent, a skin exfoliating agent, a skin peeling agent, a skin healing agent, a skin protectant agent, a skin purifier agent, a vasoconstrictor agent, a wound healing agent, or a combination thereof.

The total weight of additive(s) can be in a range from about 0.001 wt % to about 99 wt %, 0.001 wt % to about 95 wt %, or 0.001 wt % to about 90 wt % of the total weight of the skin formulation, as well as individual concentration values or sub-ranges contained within the aforementioned ranges. In some instances, the total weight of additive(s) can range from about 0.1 wt % to about 95 wt %, about 0.1 wt % to about 90 wt %, about 0.1 wt % to about 80 wt %, about 0.1 wt % to about 70 wt %, about 0.1 wt % to about 60 wt %, about 0.1 wt % to about 50 wt %, about 0.1 wt % to about 40 wt %, about 0.1 wt % to about 30 wt %, about 0.1 wt % to about 20 wt %, or about 0.1 wt % to about 10 wt % of the total weight of the skin formulation, as well as sub-ranges or individual concentration values contained within the aforementioned ranges. In some instances, the cosmetic skin formulations can be free or substantially free of any one or more of the aforementioned additives, where “substantially free” refers to the formulation including less than about 3%, 2%, 1%, 0.9%, 0.8%, 0.7%, 0.6%, 0.5%, 0.4%, 0.3%, 0.2%, or 0.1% by weight in the total weight of the skin formulation.

As noted above, the cosmetic skin formulations may contain a wide range of excipients, carriers, and/or additives. Beyond those named above, the 2012 International Cosmetic Ingredient Dictionary & Handbook, 14^{th} Edition, as well as the Cosmetic Bench Reference-Directory of Cosmetic Ingredients (published by Cosmetics & Toiletries) describes a wide variety of non-limiting cosmetic and pharmaceutical ingredients commonly used in the skin care, personal care, feminine care, and dermatology and pharmaceutical industry, which are available for use in the present formulations. Additional examples can be found in the books provided by the United States Pharmacopeia (USP), the National Formulary (NF), and other references for cosmetic and pharmaceutical ingredients known in the art. Each of these references is herein incorporated by reference in its entirety. This information is regularly updated by the addition of new ingredients.

The following are non-limiting examples of excipients and/or additives that may form part of the cosmetic skin formulations described herein.

### 1. Vitamins

The cosmetic skin formulations may contain one or more vitamins, such as ascorbates (e.g., vitamin C, vitamin C derivatives, ascorbic acid, ascorbyl glucoside, ascorbyl palmitate, magnesium ascorbyl phosphate, sodium ascorbyl phosphate, tetrahexadecyl ascorbate, ascorbyl 3-aminopropyl phosphate), vitamin B, vitamin B derivatives, vitamin 131 to vitamin 312 and theirs derivatives, vitamin K, vitamin K derivatives, vitamin H; vitamin D, vitamin D3, vitamin D derivatives, vitamin E (tocopheryl acetate), vitamin E derivatives, and pro-vitamins thereof, such as panthenol and mixtures thereof. The vitamin compounds may be included as the substantially pure material, or as an extract obtained by suitable physical and/or chemical isolation from natural (e.g., plant) sources. In one example, when vitamin compounds are present in the skin formulations, the formulations contain from about 0.0001% to about 25%, or from about 0.001% to about 10%, or from about 0.01% to about 5%, or from about 0.1% to about 1%, by total weight of the formulation, of the vitamin.

### 2. Sunscreens

The cosmetic skin formulations may optionally contain a sunscreen. As used herein, “sunscreen” includes both sunscreen agents and physical sunblocks. Suitable sunscreen actives may be organic or inorganic. A wide variety of conventional organic or inorganic sunscreen actives are suitable for use herein. In one example, the formulation contains from about 0.1% to about 25%, more typically from about 0.5% to about 10% by total weight of the formulation, of the sunscreen active. Exact amounts will vary depending upon the sunscreen chosen and the desired Sun Protection Factor (SPF). Organic UV-screening agents include, but are not limited to: ethylhexyl salicylate, butyl methoxydibenzoylmethane, ethylhexyl methoxycinnamate, octocrylene, phenylbenzimidazole sulphonic acid, terephthalylidene dicamphor sulphonic, benzophenone-3, benzophenone-4, benzophenone-5,4-methylbenzylidene camphor, benzimidazilate, anisotriazine, Ethylhexyl triazone, diethylhexyl butamido triazone, methylene bis-benzotriazolyl tetramethylbutylphenol, drometrizole trisiloxane, or a combination or mixture thereof. Exemplary inorganic sunscreen agents that may be used in the formulation include, but are not limited to, nanopigments (mean size of the primary particles: generally between 5 nm and 100 nm, or between 10 nm and 50 nm; or their aggregates) of coated or uncoated metal oxides, such as for example nanopigments of titanium oxide (amorphous or crystallized in the form of rutile and/or anatase), iron, zinc, zirconium or cerium oxides and mixtures thereof. Suitable coating agents include, but are not limited to, alumina and/or aluminum stearate, and silicones.

### 3. Anti-Wrinkle and Anti-Atrophy Agents

The cosmetic skin formulations can contain one or more anti-wrinkle or anti-atrophy agents. Exemplary anti-wrinkle/anti-atrophy agents suitable for use in the formulations include, but are not limited to, amino acids, N-acetyl derivatives of amino acids (e.g., N-acetyl-cysteine), hydroxy acids (e.g., alpha-hydroxy acids such as lactic acid and glycolic acid or beta-hydroxy acids such as salicylic acid and salicylic acid derivatives such as the octanoyl derivative, lactobionic acid), keto acids (e.g., pyruvic acid), phytic acid, ascorbic acid (vitamin C), retinoids (e.g., retinoic acid, tretinoin, isotretinoin, adapalene, retinol, retinylaldehyde, retinylpalmitate, and other retinoid derivatives), kinetin (N6-furfuryladenine), zeatin and their derivatives (e.g., furfurylamino-tetrahydropyranyladenine), niacinamide (nicotinamide); growth factors and cytokines (e.g., TGF-beta 1, 2 and 3, EGF, FGF-2, PDGF, IL-1, IL-6, IL-8, IGF-1, IGF-2, etc.), cell lysates (e.g., dermal fibroblast cell lysate, stem cell lysate, processed skin cell proteins (PSP®), etc.), conditioned cell culture mediums (e.g., conditioned cell culture medium from dermal fibroblasts, conditioned cell culture medium from stem cells (e.g., epidermal stem cells, adipose stem cells, mesenchymal stem cells, etc.); cosmetic ingredients marketed under the trade names Nouricel-MD®, TNS®, or CCM™ Complex; etc.); cell extracts, stem cell extracts, components from stem cells; ingredients stimulating epidermal or other human adult stem cells; skin conditioning agents, stilbenes, cinnamates, ingredients activating sirtuin 1 (e.g., resveratrol); ingredients improving the functioning of the mitochondria; dimethylaminoethanol, synthetic anti-aging peptides, peptides from natural sources (e.g., soy peptides), and salts of sugar acids (e.g., Mn gluconate, Zn gluconate), lipoic acid; lysophosphatidic acid, vitamin B3 (niacinamide) compounds, and other vitamin B compounds (e.g., thiamine (vitamin B1), pantothenic acid (vitamin B5), riboflavin (vitamin B2), and their derivatives and salts (e.g., HCl salts or calcium salts).

When anti-wrinkle/anti-atrophy agents are present in the skin formulations, the formulations can include from about 0.0001% to about 25%, or from about 0.001% to about 25%, or from about 0.01% to about 25%, or from about 0.001% to about 10%, or from about 0.01% to about 10%, or from about 0.01% to about 10%, or from about 0.01% to about 5%, or from about 0.1% to about 5%, or from about 0.1% to about 1%, by total weight of the formulation, of the anti-wrinkle/anti-atrophy agent(s). The exact content (%) of anti-wrinkle/anti-atrophy agents to be used in the formulations depends on the particular anti-wrinkle/anti-atrophy agent utilized since such agents can vary in potency.

### 4. Humectants, Moisturizers, and Conditioning Agents

The cosmetic skin formulations can contain a safe and effective amount of a conditioning agent. Categories of suitable conditioning agents include, for example, humectants, moisturizers, and skin conditioners. A variety of these materials can be employed and can be present in an amount ranging from about 0.01% to about 80%, or from about 0.1% to about 80%, or from about 0.1% to about 50%, or from about 0.1% to about 25%, or from about 0.5% to about 25%, or from about 0.5% to about 10%, by weight of the formulation. The exact content (%) of humectants, moisturizers, and conditioning agents to be used in the formulations depends on the humectant, moisturizer, and conditioning agent utilized since such agents can vary in potency.

Humectants are ingredients that help maintain moisture levels in skin. Exemplary suitable conditioning agents include, but are not limited to, glycerin, hydrolyzed sodium hyaluronate, hydrolyzed vegetable protein, phytosphingosine, sodium hyaluronate crosspolymer, hydroxyacetophenone, polyhydric alcohols, water soluble alkoxylated nonionic polymers, and mixtures thereof. Polyhydric alcohols useful in the formulations described herein include polyhydroxy alcohols aforementioned and glycerin, hexylene glycol, ethoxylated glucose, 1,2-hexane diol, dipropylene glycol, trehalose, diglycerin, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof. Water soluble alkoxylated nonionic polymers useful in the formulations described herein include polyethylene glycols and polypropylene glycols having a molecular weight of up to about 1000 Da, such as those with CTFA names PEG-200, PEG-400, PEG-600, PEG-1000, and mixtures thereof. Additional suitable humectants include, but are not limited to, sodium hyaluronate, sodium acetylated hyaluronate, acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed proteins, hydroxypropyl trimonium hyaluronate, inositol, lactitol, maltitol, maltose, mannitol, mannose, methoxy polyethylene glycol, myristamidobutyl guanidine acetate, polyglyceryl sorbitol, potassium pyrollidone carboxylic acid (PCA), propylene glycol, butylene glycol, sodium pyrollidone carboxylic acid (PCA), sorbitol, sucrose, dextran sulfate (i.e., of any molecular weight), natural moisturizing factors, and/or urea, and combinations thereof.

Suitable skin conditioners include, but are not limited to, guanidine, urea, glycolic acid, glycolate salts (e.g., ammonium and quaternary alkyl ammonium), salicylic acid, lactic acid, lactate salts (e.g., ammonium and quaternary alkyl ammonium), aloe vera in any of its variety of forms (e.g., aloe vera gel), polyhydroxy alcohols such as sorbitol, mannitol, xylitol, erythritol, hexanetriol, butanetriol, propylene glycol, butylene glycol, hexylene glycol, hydrogenated ethylhexyl olivate, Camellia japonica seed oil, hydroxyapatite, hydrogenated olive oil unsaponifiables, Sesamum indicum (sesame) seed oil, caprylyl glycol, proline, pentylene glycol, and the like, polyethylene glycols, propoxylated glycerols, sugars (e.g., melibiose), starches, sugar and starch derivatives (e.g., alkoxylated glucose, fructose, glucosamine), C₁-C₃₀ monoesters and polyesters of sugars and related materials, hyaluronic acid, lactamide monoethanolamine, acetamide monoethanolamine, panthenol, dexpanthenol, allantoin, and mixtures thereof. Suitable skin conditioners also include fatty acids, fatty acid esters, lipids, ceramides, cholesterol, cholesterol esters, bee wax, petrolatum, and mineral oil.

### 5. Emollients

One or more emollients may be included in the cosmetic skin formulations described herein. An emollient generally refers to an ingredient that can help skin maintain a soft, smooth, and pliable appearance. Emollients typically remain on the skin surface, or in the stratum corneum, and act as a moisturizer, or lubricant and reduce flaking. Some examples of emollients include, but are not limited to, hydrogenated ethylhexyl olivate, isononyl isononanoate, caprylic/capric triglyceride, cetearyl alcohol, hydrogenated lecithin, stearic acid, cholesterol, polyglyceryl-4 caprate, glycosphingolipids, hydrogenated polydecene, acetyl arginine, acetylated lanolin, algae extract, apricot kernel oil polyethylene glycol-6 esters, avocado oil polyethylene glycol-11 esters, bis-polyethylene glycol-4 dimethicone, butoxyethyl stearate, glycol esters, alkyl lactates, caprylyl glycol, cetyl esters, cetyl laurate, coconut oil polyethylene glycol-10 esters, alkyl tartrates, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, palm oil, coconut oil, myreth-3 palmitate, octyldecanol, octyldodecanol, Odontella aurita oil, 2-oleamido-1,3 octadecanediol, palm glycerides, polyethylene glycol avocado glycerides, polyethylene glycol castor oil, polyethylene glycol-22/dodecyl glycol copolymer, polyethylene glycol shea butter glycerides, phytol, raffinose, stearyl citrate, sunflower seed oil glycerides, petrolatum, silicon oils including but not limited to caprylyl methicone, and/or tocopheryl glucoside.

### 6. Anti-Oxidants and Radical Scavengers

The cosmetic skin formulation optionally includes an anti-oxidant/radical scavenger. In some instances, the formulation contains from about 0.001% to about 25%, or from about 0.01% to about 10%, or from about 0.1% to about 5%, of an anti-oxidant/radical scavenger. The exact content (%) of anti-oxidant/radical scavengers in the formulations depends on the particular anti-oxidant/radical scavenger utilized since such agents can vary in potency.

Suitable anti-oxidants/radical scavengers include, but are not limited to, ascorbic acid (vitamin C) and its salts, ascorbyl esters of fatty acids, and other ascorbic acid derivatives (e.g., magnesium ascorbyl phosphate, sodium ascorbyl phosphate, ascorbyl sorbate, ascorbyl palmitate, tetrahexyldecyl ascorbate, etc.), tocopherol (vitamin E), tocopherol sorbate, tocopherol acetate, other esters of tocopherol, beta-carotene, butylated hydroxy benzoic acids and their salts, ferulic acid, peroxides including hydrogen peroxide, perborate, thioglycolates, persulfate salts, 6-hydroxy-2,5,7,8-tetramethylchroman-2-carboxylic acid (commercially available under the trade name Trolox™), gallic acid and its alkyl esters, especially propyl gallate, uric acid and its salts and alkyl esters, amines (e.g., N,N-diethylhydroxylamine, amino-guanidine), nordihydro guaiaretic acid, bioflavonoids, sulfhydryl compounds (e.g., glutathione), dihydroxy fumaric acid and its salts, lysine pidolate, arginine pilolate, amino acids, silymarin, lysine, 1-methionine, proline, superoxide dismutase, sorbic acids and its salts, lipoic acid, olive extracts, tea extracts, resveratrol, polyphenols such as proanthocyanidine from pine bark, carotenoids, curcumin compounds such as tetrahydrocurcumin, coenzyme Q10, OCTA (L-2-oxo-4-thiazolidine carboxylic acid), selenium, creatine, glutathione, N-acetyl cysteine, N-acetyl cysteine esters, dimethylmethoxy chromanol, lipoic acid, melanin; plant extracts containing polyphenols including but not limited to coffee berry extracts, green tea extracts, rosemary extracts, witch hazel extracts, Zingiber officinale (ginger) root extract, and grape skin/seed extracts, may be used.

### 7. Antimicrobial Peptide Sequestering Agents

Antimicrobial peptide sequestering agents may form part of the cosmetic skin formulation. Suitable antimicrobial peptide sequestering agents include, without limitation, a sulfated or polysulfated monosaccharide, and salts and complexes thereof; a sulfated or polysulfated disaccharide, and salts and complexes thereof; a sulfated or polysulfated polysaccharide, and salts and complexes thereof; a dextran sulfate (e.g., sodium dextran sulfate), and salts and complexes thereof; chondroitin sulfate, and salts and complexes thereof; pentosan polysulfate, and salts and complexes thereof; sucrose sulfate (e.g., any sucrose sulfate such as sucrose octasulphate other than aluminum sucrose sulfate), and salts and complexes thereof; a fucoidan, and salts and complexes thereof; a sulfated galactan, and salts and complexes thereof; a carrageenans (e.g., Chondrus crispus), and salts and complexes thereof; starch sulfate, and salts and complexes thereof; cellulose sulfate, and salts and complexes thereof; a sulfated glycosaminoglycan, and salts and complexes thereof; a heparin; a heparan sulfate; sulfated glucan; or any combinations thereof. The antimicrobial peptide sequestering agent may be a plant extract, an algae extract, an aloe vera (barbadensis) extract, a cactus extract, or a shark or fish cartilage extract. The antimicrobial peptide sequestering compound may also be a sulfated or polysulfated polymer (e.g., poly(vinyl sulfate), poly(anethole sulfonate)). Suitable polymeric sulfonic acid that can be used in the methods and formulations described herein are hydrophobically modified polymeric sulfonic acids, such as ARISTOFLEX® HMP or ARISTOFLEX® AVC (Clariant). Alternatively, the antimicrobial peptide sequestering agent is a phosphate or polyphosphate (e.g., a monosaccharide phosphate, a disaccharide phosphate, a polysaccharide phosphate, a glycerophosphate salt, or a starch phosphate). Suitable examples of starch phosphates include, but are not limited to hydroxypropyl starch phosphates (i.e., Structure XL (National Starch, LCC)). The antimicrobial peptide sequestering agent may also be a phospholipid such as phosphatidylcholine or lecithin. Further, the antimicrobial peptide sequestering compound can be a carboxylate, a polyhydroxy acid, hyaluronic acid, alginate, and/or polylactic acid. In some instances, the antimicrobial peptide sequestering compounds are between 100 to 10,000 g per mol.

### 8. Rosacea Inhibitory Agents and α-Adrenergic Receptor Agonists

The cosmetic skin formulation optionally includes a rosacea inhibitory agent. Suitable rosacea inhibitory agents include, but are not limited to, metronidazole, sulfacetamide, sodium sulfacetamide, sulfur, dapson, doxycycline, minocycline, clindamycin, clindamycin phosphate, erythromycin, tetracylclines, azelaic acid, calcium dobesilate, maleic acid, and any compatible combinations thereof); α-adrenergic receptor agonists (e.g., clonidine, amphetamine, doxtroamphetamine, apraclonidine, dipivefrin, α-methyldopa, oxymetazoline, oxymetazoline hydrochloride, methoxamine, metaraminol, medetomidine, dexmedetomidine, ethylnorepinephrine, guanfacine, guanabenz, phenylephrine, phenylephrine hydrochloride, ephedrine, epinine, epinephrine, ethylnorepinephrine, levarterenol, lofexidine, norepinephrine, norphenylephrine, norephedrine, phenylpropanolamine, pemoline, propylhexadrine, pseudoephedrine, methamphetamine, methylnorepinephrine, methylphenidate, mephentermine, midodrine, mivazerol, moxonidine, desglymidodrine, tetrahydrozoline, tetrahydrozoline hydrochloride, cirazoline, amidephrine, brimonidine, brimonidine tartrate, naphazoline, isoproterenol, xylazine, xylometazoline, and/or tizanidine); chemicals and botanical extracts with vasoconstrictor properties including, but not limited to, corticosteroids, ephedrine, pseudoephedrine, caffeine, and/or escin; ephedra, Phedra sinica, Hamamelis viginiana, Hydrasiis canadensis, Lvcopus virginicus, Aspidosperma quebracho, Cytisus scoparius, Raphanus sativus linn (radish leave extracts), horse chestnut extracts, etc., as well as any compatible combinations thereof.

### 9. Skin Lightening Agents, and Skin Bleaching Agents

The cosmetic skin formulations optionally contain a skin lightening agent. Suitable skin lightening agents include, but are not limited to, ascorbic acid and derivatives thereof; kojic acid and derivatives thereof; resorcinol and derivatives thereof (including but not limited to 4-ethyl resorcinol, 4-butyl resorcinol, 4-hexyl resorcinol, 4-octyl resorcinol, 4-decyl resorcinol, 6-methyl resorcinol, 6-ethyl resorcinol, 6-butyl resorcinol, 6-hexyl resorcinol, 6-octyl resorcinol, 6-decyl resorcinol, 4-phenylethyl resorcinol), retinoic acid and derivatives thereof (e.g., retinol, retinyl palmitate), L-leucine and derivatives thereof (e.g., N-acyl derivatives of L-leucine, esters of L-leucine, etc.), glycine and derivatives thereof, disodium glycerophosphate and derivatives thereof, undecenoyl phenylalanine, arbutin and derivatives thereof (e.g., dehydroxyarbutin), niacinamide and derivatives thereof, hydroquinone; mequinol, glabridin, aleosin, curcumin, genistein, ethyl linoleate, tranexaminic acid, azelaic acid, resveratrol and derivatives thereof (e.g., oxyresveratrol), N-acetyl glucosamine, 4-isopropylcetchol, 4-ethoxybenzaldehyde, 2-ethoxybenzaldehyde, 4-propoxybenzaldehyde, alpha-hydroxyacids (e.g., glycolic acid, lactic acid, etc.), salicylic acid, polyphenols; and/or various plant extracts, such as those from licorice, grape seed, mulberry, soy, green tea, and/or bear berry; and/or any combination thereof.

When used, the cosmetic skin formulations may contain from about 0.01% to about 15%, or from about 0.1% to about 10%, or from about 0.5% to about 5%, by weight of the formulation, of a skin lightening agent. The exact content (%) of skin lightening agents to be used in the formulations depends on the particular skin lightening agent utilized since such agents can vary in potency.

### 10. Skin Protectants

Suitable skin protectant agents for use in the cosmetic skin formulations described herein include, for example, a compound that protects injured or exposed skin or mucous membrane surfaces from harmful or irritating external compounds. Representative examples include algae extract, allantoin, Camellia sinensis leaf extract, cerebrosides, dimethicone, glucuronolactone, glycerin, kaolin, lanolin, malt extract, mineral oil, petrolatum, white petrolatum, potassium gluconate, colloidal oat meal, calamine, cocoa butter, starch, zinc oxide, zinc carbonate, zinc acetate, and/or talc.

### 11. Desquamation Agents, Keratolytic Agents, and Peeling Agents

A desquamation/keratolytic active may be included in the cosmetic skin formulations described herein. In some instances, the formulation contain from about 0.01% to about 30%, or from about 0.1% to about 10%, or from about 0.5% to about 5%, by total weight of the formulation, of a desquamating/keratolytic agent. The exact content (%) of desquamating/keratolytic agents to be used in the formulations depends on the particular desquamating/keratolytic agent utilized since such agents can vary in potency.

Examples of suitable keratolytic and/or desquamating agents include, but are not limited to, urea, salicylic acid and alkyl derivatives thereof, saturated and unsaturated monocarboxylic acids, saturated and unsaturated bicarboxylic acids, tricarboxylic acids, alpha hydroxyacids and beta hydroxyacids of monocarboxylic acids, alpha hydroxyacids and beta hydroxyacids of bicarboxylic acids, alpha hydroxyacids and beta hydroxyacids of tricarboxylic acids, ketoacids, alpha ketoacids, beta ketoacids, of the polycarboxylic acids, of the polyhydroxy monocarboxylic acids, of the polyhydroxy bicarboxylic acids, of the polyhydroxy tricarboxylic acids. Resorcinol and its low-molecular weight derivatives are other examples of useful keratolytic and/or desquamating agents.

In certain instances, the keratolytic agents include, but are not limited to, glycolic acid, tartaric acid, salicylic acid, citric acid, lactic acid, pyruvic acid, gluconic acid, glucuronic acid, malic acid, mandelic acid, oxalic acid, malonic acid, succinic acid, acetic acid, phenol, resorcinol, retinoic acid, adapalene, trichloroacetic acid, 5-fluoro uracil, or azelaic acid, or a combination thereof. Keratolytic agents are also the salts, esters, possible cis- or trans-forms, racemic mixtures and/or the relative dextrorotatory or levorotatory forms of the above listed compounds. Such substances can be used singularly or in combination with each other.

### 12. Anti-Inflammatory Agents

An anti-inflammatory agent may be included in the cosmetic skin formulations described. In some instances, an anti-inflammatory agent is included in the formulation at a concentration of from about 0.01% to about 10%, or from about 0.5% to about 5%, by weight of the formulation. The exact content (%) of anti-inflammatory agents to be used in the formulations depends on the particular anti-inflammatory agent utilized since such agents can vary in potency.

Steroidal anti-inflammatory agents can include, but are not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, fluradrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used.

In addition, non-steroidal anti-inflammatory agents can be used. The varieties of compounds encompassed by this group are well-known to those skilled in the art. Specific non-steroidal anti-inflammatory agents that can be useful in the formulation can include, but are not limited to, diclofenac, indomethacin, oxicams such as piroxicam, salicylates such as aspirin; acetic acid derivatives such as felbinac, fenamates such as etofenamate, flufenamic acid, mefenamic acid, meclofenamic acid, tolfenamic acid; propionic acid derivatives such as ibuprofen, naproxen, pyrazoles, and mixtures thereof. Mixtures of these non-steroidal anti-inflammatory agents may also be employed, as well as the dermatologically acceptable salts and esters of these agents. For detailed disclosure of the chemical structure, synthesis, side effects, etc. of non-steroidal anti-inflammatory agents, one may refer to standard texts, including Anti-inflammatory and Anti-Rheumatic Drugs, K. D. Rainsford, Vol. I-111, CRC Press, Boca Raton, (1985), and Anti-inflammatory Agents, Chemistry and Pharmacology, 1, R. A. Scherrer, et al., Academic Press, New York (1974).

Finally, “natural” anti-inflammatory agents can be useful in skin formulations described. Such agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), kola extract, chamomile, red clover extract, sea whip extract, licorice extract, and tea extract may be used.

Anti-inflammatory agents useful herein include allantoin and compounds of the Licorice, including glycyrrhetic acid, glycyrrhizic acid, and derivatives thereof (e.g., salts and suitable esters). Additional anti-inflammatory agents include diosgenol, saponines, sapogenines, lignanes, triterpenes saponosides and genines.

Additional examples of anti-inflammatory agents can include tetraacetylphytosphingosine; anti-inflammatory interleukins (e.g., IL-1ra, IL-6, IL-8, IL-10); anti-inflammatory fatty acids (e.g., linoleic acid, linolenic acid) and their derivatives (e.g., esters), isoprenylcystein analogues (i.e., N-acetyl-S-farnesyl-L-cysteine), aromatic aldehydes with anti-inflammatory properties (e.g., 4-methoxy benzaldehyde, 4-ethoxy benzaldehyde, 4-butoxy benzaldehyde, 4-penthoxy benzaldehyde), as well as any compatible combinations thereof.

### 13. Anti-Acne Agents

The cosmetic skin formulations can contain one or more anti-acne agents. Examples of useful anti-acne actives include, but are not limited to, resorcinol, sulfur, erythromycin, salicylic acid, benzoyl peroxide, retinoic acid, tretinoin, alpha-hydroxy acids (e.g., glycolic acid, lactic acid), dehydroacetic acid and zinc. When anti-acne compounds are present in the formulation, the formulation can contain from about 0.0001% to about 50%, or from about 0.001% to about 20%, or from about 0.01% to about 10%, or from about 0.1% to about 5%, by total weight of the formulation, of the anti-acne agent. The exact content (%) of anti-acne agents to be used in the formulations depends on the particular antimicrobial, anti-bacterial and anti-acne active utilized since such agents can vary in potency.

### 14. Antimicrobial, Anti-Bacterial, and Anti-Fungal Agents

The cosmetic skin formulations can contain one or more anti-fungal or anti-microbial agents. A safe and effective amount of an antimicrobial or antifungal active can be added to the present formulations. For example, the formulation can contain from about 0.001% to about 10%, or from about 0.01% to about 5%, or from about 0.05% to about 2%, by total weight of the formulation, of an antimicrobial or antifungal agent. The exact content (%) of antimicrobial, anti-bacterial and anti-fungal actives to be used in the formulations depends on the particular antimicrobial, anti-bacterial and anti-fungal active utilized since such agents can vary in potency.

Suitable anti-microbial agents include, but are not limited coal to tar, sulfur, aluminum chloride, gentian violet, octopirox (piroctone olamine), 3,4,4′-trichlorocarbanilide (trichlosan), triclocarban, ciclopirox olamine, undecylenic acid and it's metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, iodopropynyl butylcarbamate, azelaic acid, isothiazalinones such as octyl isothiazolinone and azoles, parabens (e.g., methylparaben, ethylparaben, etc.), glycols (e.g., hexylenglycol, ethylhexylglycerin), and combinations thereof.

For example, suitable agents with anti-fungal properties are ketoconazole, naftifine hydrochloride, oxiconazole nitrate, sulconazole nitrate, urea, terbinafine hydrochloride, selenium sulfide.

### 15. Anesthetics

The cosmetic skin formulations may also contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol; and pharmaceutically acceptable salts thereof; benzyl alcohol, camphor, menthol, resorcinol; and appropriate combinations thereof.

### 16. Plant Extracts and Vegetable Extracts

The skin formulations may also contain a safe amount of a plant extract and vegetable extract. Examples of plant or vegetable extracts include extracts obtained from ivy (in particular English Ivy (Hedera helix)), Chinese thorowax (Bupleurum chinensis), barley, Bupleurum falcatum, arnica (Arnica montana L), rosemary (Rosmarinus officinalis N), marigold (Calendula officinalis), sage (Salvia officinalis L), soy, ginseng (Panax ginseng), Ginko biloba, St.-John's-Wort (Hyperycum perforatum), butcher's-broom (Ruscus aculeatus L), European meadowsweet (Filipendula ulmaria L), big-flowered Jarva tea (Orthosiphon stamincus benth), algae (Fucus vesiculosus), birch (Betula alba), green tea, white tea, fermented tea, cola nuts (Cola nipida), horse-chestnut, bamboo, spadeleaf (Centella asiatica), heather, fucus, willow, witch hazel, wild yam, mouse-ear, escine, cangzhu, chrysanthellum indicum, plants of the Armeniacea genus, Atractylodis platicodon, Sinnomenum, Pharbitidis, Flemingia, Coleus such as C. forskohlii, C. blumei, C. esquirolii, C. scutellaroides, C. xanthantus and C. barbatus, root of Coleus barbatus, Ballote, Guioa, Davallia, Terminalia, Barringtonia, Trema, antirobia, cecropia, argania, dioscoreae such as Dioscorea opposita or Mexican, Ammi visnaga, Centella asiatica and Siegesbeckia, in particular Siegesbeckia orientalis, the family of Ericaceae in particular bilberry extracts (Vaccinium angustifolium) or Arctostaphylos uva ursi, aloe vera, plant sterols (e.g., phytosterol), Manjistha (extracted from plants in the genus Rubia, particularly Rubia Cordifolia), and Guggal (extracted from plants in the genus Commiphora, particularly Commiphora mukul), kola extract, chamomile, red clover extract, Piper methysticum, Bacopa monieri extract, sea whip, Glycyrrhira glabra, mulberry, melaleuca (tea tree), mushroom extracts, Larrea divaricata, Rabdosia rubescens, Euglena gracilis, Fibraurea recisa Hirudinea, Chaparral sorghum, sun flower extract, Enantia chlorantha, Mitracarpe of Spermacocea genus, Buchu barosma, Lavsonia inermis L., adiantium Capillus-Veneris L., Chelidonium majus, Luffa cylindrical, Japanese Mandarin (Citrus reticulata Blanco var. unshiu), broccoli extract, Camelia sinensis, Imperata cylindrical, Glaucium flavum, Cupressus Sempervirens, Polygonatum multiflorum, loveyly hemsleya, Sambucus nigra, Phaseolus huatus, Centaurium, Macrocystis pyrifera, Turnera diffusa, Anemarrhena asphodeloides, Portulaca pilosa, Humulus lupulus, Coffee Arabica, coffee berry, black berry, Ilex paraguariensis; and the like.

### 17. Oils and Lipids

The cosmetic skin formulations can contain any cosmetic or dermatological oil or a mixture thereof. Examples of such oils include but are not limited to aliphatic hydrocarbons such as liquid paraffin, squalene, squalane, vaseline and ceresin; silicon oils such as dimethicone and cyclomethicones; vegetable oils such as avocado oil, apricot oil, almond oil, borage oil, borage seed oil, camellia oil, canola oil, castor oil, coconut oil, cocoa butter, corn oil, cottonseed oil, olive oil, evening primrose oil, flax seed oil, palm oil, palm kernel oil, peanut oil, rapeseed oil, safflower oil, sesame oil, sweet almond oil, rose hip oil, calendula oil, chamomile oil, eucalyptus oil, juniper oil, safflower oil, sandalwood oil, tea tree oil, sunflower oil, soybean oil, wheat germ oil; animal oils such as shark liver oil, cod liver oil, whale oil, beef tallow and butterfat; waxes such as beeswax, carnauba palm wax, spermaceti and lanolin; fatty acids such as lauric acid, myristic acid, palmitic, acid, stearic acid, oleic acid, behenic acid; omega-3 fatty acids such as alpha-linolenic acid, eicosapentaenoic acid, and docosahexaenoic acid; omega-6 fatty acids such as linoleic acid and gamma-linolenic acid; aliphatic alcohols such as lauryl, stearyl, cetyl, and oleyl alcohol; and aliphatic esters such as isopropyl, isocetyl, or octadecyl myristate, butyl stearate, hexyl laureate, diisopropyl ester of adipic acid, or diisopropyl sebacate; and/or mixtures thereof. Generally, the oils are refined and/or hydrogenated. Lipids include monoglycerides, diglycerides, triglycerides, phospholipids, and ceramides.

### 18. Suspending Agents

The cosmetic skin formulations may contain a suspending agent, such as at concentrations effective for suspending water-insoluble material in dispersed form in the formulations or for modifying the viscosity of the formulation. Such concentrations can range from about 0.1% to about 10%, or from about 0.25% to about 5.0%. Suspending agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, nitro cellulose, sodium carboxymethyl cellulose, crystalline cellulose, cellulose powder, polyvinylpyrrolidone, polyvinyl alcohol, guar gum, hydroxypropyl guar gum, arabia gum, galactan, carob gum, pectin, agar, starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid. The aforementioned thickening agents can also be used herein as suspending agents.

Other optional suspending agents include crystalline suspending agents which can be categorized as acyl derivatives, long chain amine oxides, long chain acyl derivatives and mixtures thereof. Exemplary suspending agents can include ethylene glycol esters of fatty acids, alkanol amides of fatty acids, long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin). Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

19. Emulsifying Agents (Surfactants) Emulsifying agents include a wide variety of nonionic, cationic, anionic, zwitterionic, and amphoteric surfactants, such as are known in the art and discussed below, and may be included in the cosmetic skin formulations described.

Hydrophilic surfactants (cationic, anionic, zwitterionic, amphoteric) useful herein can contain a single surfactant, or any combination of suitable surfactants. The exact surfactant (or surfactants) chosen depends on the pH of the formulation and the other components present.

Useful nonionic surfactants include the condensation products of alkylene oxides with fatty acids (i.e., alkylene oxide esters of fatty acids), the condensation products of alkylene oxides with 2 moles of fatty acids (i.e., alkylene oxide diesters of fatty acids), the condensation products of alkylene oxides with fatty alcohols (i.e., alkylene oxide ethers of fatty alcohols), the condensation products of alkylene oxides with both fatty acids and fatty alcohols [i.e., wherein the polyalkylene oxide portion is esterified on one end with a fatty acid and etherified (i.e., connected via an ether linkage) on the other end with a fatty alcohol]. Nonlimiting examples of these alkylene oxide derived nonionic surfactants include ceteth-6, ceteth-10, ceteth-12, ceteareth-6, ceteareth-10, ceteareth-12, steareth-6, steareth-10, steareth-12, steareth-21, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof. Still other useful nonionic surfactants include polyhydroxy fatty acid amide surfactants. Suitable nonionic surfactants include, but are not limited to, steareth-21, ceteareth-20, ceteareth-12, sucrose cocotte, steareth-100, PEG-100 stearate, and mixtures thereof. Other nonionic surfactants suitable for use in the formulations disclosed herein include sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, and mixtures thereof. Nonlimiting examples of these emulsifiers include: glyceryl stearate, sodium polyacryloyldimethyl taurate, polyglyceryl-3 cetyl ether, polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, sorbitan isostearate, polyoxyethylene 4 lauryl ether sodium stearate, polyglyceryl-4 isostearate, hexyl laurate, PPG-2 methyl glucose ether distearate, PEG-100 stearate, and mixtures thereof. Another group of non-ionic surfactants useful herein are fatty acid ester blends based on a mixture of sorbitan or sorbitol fatty acid ester and sucrose fatty acid ester, the fatty acid in each instance being a C₈-C₂₄ or C₁₀-C₂₀.

Also useful herein are cationic surfactants, such as dialkyl quaternary ammonium compounds. Non-limiting examples of these cationic emulsifiers include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof. Nonlimiting examples of quaternary ammonium salt cationic surfactants include those selected from cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, and mixtures thereof. Additional quaternary ammonium salts include those wherein the C₁₂ to C₃₀ alkyl carbon chain is derived from a tallow fatty acid or from a coconut fatty acid. The term “tallow” refers to an alkyl group derived from tallow fatty acids (usually hydrogenated tallow fatty acids), which generally have mixtures of alkyl chains in the C₁₆ to C₁₈ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, and mixtures thereof. An example of a quaternary ammonium compound having an alkyl group with an ester linkage is ditallowyl oxyethyl dimethyl ammonium chloride. Additional cationic surfactants are those selected from behenamidopropyl PG dimonium chloride, dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, stearamidopropyl PG-dimonium chloride phosphate, stearamidopropyl ethyldiammonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof. In some instances, the cationic surfactants are those selected from behenamidopropyl PG dimonium chloride, dilauryl dimethyl ammonium chloride, distearyl dimethyl ammonium chloride, dimyristyl dimethyl ammonium chloride, dipalmityl dimethyl ammonium chloride, and mixtures thereof.

A wide variety of anionic surfactants can also be used. Nonlimiting examples of anionic surfactants include the alkoyl isethionates, and the alkyl and alkyl ether sulfates. The reaction products of fatty acids esteritied with isethianonic acid and neutralized, i.e., the alkoyl isethionates typically have the formula RCOOCH₂CH₂SO₃M wherein R is alkyl or alkenyl of from about 10 to about 30 carbon atoms, and M is a water-soluble cation such as ammonium, sodium, potassium and triethanolamine. For example, the fatty acids are derivated from coconut or palm kernel oil. Nonlimiting examples of these isethionates include those alkoyl isethionates selected from ammonium cocoyl isethionate, sodium cocoyl isethionate, sodium lauroyl isethionate, sodium stearoyl isethionate, and mixtures thereof. Also suitable are salts of fatty acids, amids of methyl taurides. The alkyl and alkyl ether sulfates typically have the respective formulae ROSO₃M and RO(C₂H₄O)ₓSO₃M, wherein R is alkyl or alkenyl of from about 10 to about 30 carbon atoms, x is from about 1 to about 10, and M is a water-soluble cation such as ammonium, alkanolamines such as triethanolamine, monovalent metals, such as sodium and potassium, and polyvalent metal cations such as magnesium and calcium. In some instances, R has from about 8 to about 18 carbon atoms, or from about 10 to about 16 carbon atoms, or from about 12 to about 14 carbon atoms, in both the alkyl and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. Such alcohols are reacted with between about 0 and about 10, or from about 2 to about 5, or about 3, molar proportions of ethylene oxide, and the resulting mixture of molecular species having, for example, an average of 3 moles of ethylene oxide per mole of alcohol, is sulfated and neutralized. Another suitable class of anionic surfactants are the water-soluble salts of the organic, sulfuric acid reaction products of the general formula R₁—SO₃-M, wherein R₁ is chosen from the group including a straight or branched chain, saturated aliphatic hydrocarbon radical having from about 8 to about 24, or about 10 to about 16, carbon atoms; and M is a cation described hereinbefore. Still other anionic synthetic surfactants include the class designated as succinamates, olefin sulfonates having about 12 to about 24 carbon atoms, and beta-alkyloxy alkane sulfonates. Examples of these materials are sodium lauryl sulfate and ammonium lauryl sulfate. Other anionic surfactants suitable for use in the formulations are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid. Other suitable anionic surfactants include olefin sulfonates having about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. Another class of anionic surfactants suitable for use in the formulations is the beta-alkyloxy alkane sulfonate class. Other anionic materials useful herein are soaps (i.e., alkali metal salts, e.g., sodium or potassium salts) of fatty acids, typically having from about 8 to about 24 carbon atoms, or from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared.

Amphoteric and zwitterionic surfactants can also be included herein. Examples of amphoteric and zwitterionic surfactants which can be used in the formulations are those which are broadly described as derivatives of aliphatic secondary and tertiary amines in which the aliphatic radical can be straight or branched chain and wherein one of the aliphatic substituents contains from about 8 to about 22 carbon atoms (or C₈-C₁₈) and one contains an anionic water solubilizing group, e.g., carboxy, sulfonate, sulfate, phosphate, or phosphonate. Examples are alkyl imino acetates, and iminodialkanoates and aminoalkanoates of the formulas RN[CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Exemplary amphoteric surfactants for use in the formulations can include cocoamphoacetate, cocoamphodiacetate, lauroamphoacetate, lauroamphodiacetate, and mixtures thereof. Also included are Imidazolinium and ammonium derivatives. Specific examples of suitable amphoteric surfactants include sodium 3-dodecyl-aminopropionate, sodium 3-dodecylaminopropane sulfonate, N-alkyltaurines such as the one prepared by reacting dodecylamine with sodium isethionate; N-higher alkyl aspartic acids; and the products sold under the trade name “Miranol”. Other examples of useful amphoterics include phosphates, such as coamidopropyl PG-dimonium chloride phosphate (commercially available as Monaquat PTC, from Mona Corp.).

Zwitterionic surfactants suitable for use in the formulations are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonzaine 16SP from Lonza Corp.), lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and amidobetaines and amidosulfobetaines (wherein the RCONH(CH₂)₃ radical is attached to the nitrogen atom of the betaine), oleyl betaine (available as amphoteric Velvetex OLB-50 from Henkel), and cocamidopropyl betaine (available as Velvetex BK-35 and BA-35 from Henkel). Other useful amphoteric and zwitterionic surfactants include the sultaines and hydroxysultaines such as cocamidopropyl hydroxysultaine (available as Mirataine CBS from Rhone-Poulenc), and the alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and trialkanolamine (e.g., triethanolamine).

### 20. Thickening Agents

Thickening agents suitable for inclusion in the cosmetic skin formulations described herein include those agents commonly used as an excipient or a carrier for topical application to increase the viscosity of the formulation. Thickening agents may also be used to improve the stability of the formulation.

More specifically, such examples include but are not limited to, xanthum gum, acrylamides copolymer, agarose, amylopectin, bentonite, calcium alginate, calcium carboxymethyl cellulose, carbomer, carboxymethyl chitin, cellulose gum, dextrin, gelatin, hydrogenated tallow, hydroxylethyl-cellulose, hydroxypropylcellulose, hydroxypropyl starch, magnesium alginate, methylcellulose, microcrystalline cellulose, pectin, various polyethylene glyco″s, polyacrylic acid, polyacrylate crosspolymer-6, poly-methacrylic acid, polyvinyl alcohol, various polypropylene glycols, sodium acrylates copolymer, sodium carrageenan, xanthan gum, and/or yeast beta-glucan.

Moreover, carboxylic acid polymers can be useful thickening agents. Carboxylic acid polymers are cross-linked compounds containing one or more monomers derived from acrylic acid, substituted acrylic acids, and salts and esters of these acrylic acids and the substituted acrylic acids, wherein the cross-linking agent contains two or more carbon-carbon double bonds and is derived from a polyhydric alcohol. Examples of commercially available carboxylic acid polymers that are useful thickeners for use in the formulations described herein include the carbomers, which are homopolymers of acrylic acid cross-linked with allyl ethers of sucrose or pentaerytritol. The carbomers are available as the CARBOPOL®900 series from B.F. Goodrich (e.g., CARBOPOL® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the cross-linking agent is an allyl ether of sucrose or pentaerytritol. These copolymers are known as acrylates/C₁₋₃₀ alkyl acrylate crosspolymers and are commercially available as CARBOPOL®1342, CARBOPOL® 1382, Pemulen TR-1, and Pemulen TR-2, from B.F. Goodrich. Examples of carboxylic acid polymer thickeners which are useful herein include those selected from carbomers, acrylates/C₁₀₋₃₀ alkyl acrylate crosspolymers, and mixtures thereof.

Additional suitable thickening agents can include ammonium acryloyl dimethyltaurate/VP copolymer, SEPIMAX ZEN (polyacrylate crosspolymer-6), and/or SEPINOV WEO (hydroxyethyl acrylate/sodium acryloyldimethyl taurate copolymer).

Moreover, a wide variety of polysaccharides are useful herein as thickening agents. Non-limiting examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroxyalkylated (such as hydroxyethylated or hydroxypropylated) to form a hydroxyalkylated cellulose which is then further modified with a C₁₀₋₃₀ straight chain or branched chain alkyl group through an ether linkage. Typically these polymers are ethers of C₁₀₋₃₀ straight or branched chain alcohols with hydroxyalkylcelluloses. Examples of alkyl groups useful herein include those selected from stearyl, isostearyl, lauryl, myristyl, cetyl, isocetyl, cocoyl (e.g., alkyl groups derived from the alcohols of coconut oil), palmityl, oleyl, linoleyl, linolenyl, ricinoleyl, behenyl, and mixtures thereof. Among the alkyl hydroxyalkyl cellulose ethers is the material given the CTFA designation cetyl hydroxyethylcellulose, which is the ether of cetyl alcohol and hydroxyethylcellulose. This material is sold under the trade name NATROSOL® CS Plus from Aqualon Corporation (Wilmington, Del.). Additional examples can be found in The International Cosmetic Ingredient Dictionary and Handbook, the Cosmetic Bench Reference-Directory of Cosmetic Ingredients, the books provided by the United States Pharmacopeia (USP) and the National Formulary (NF), and other references for cosmetic and pharmaceutical ingredients known in the art. Other useful polysaccharides include scleroglucans which are a linear chain of (1-3) linked glucose units with a (1-6) linked glucose every three units, a commercially available example of which is Clearogel™ CS11 from Michel Mercier Products Inc. (Mountainside, N.J.).

Other thickening and gelling agents useful herein include materials which are primarily derived from natural sources. Non-limiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyl trimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, dextran sulfate, sodium carrageenan, tragacanth gum, xanthan gum, and/or mixtures thereof. In addition, the formulations can also optionally contain polyacrylamide polymers, especially nonionic polyacrylamide polymers including substituted branched or unbranched polymers. Among these polyacrylamide polymers is the nonionic polymer given the CTFA designation polyacrylamide and isoparaffin and laureth-7, available under the trade name Sepigel 305 from Seppic Corporation (Fairfield, N.J.). Other polyacrylamide polymers useful herein include multi-block copolymers of acrylamides and substituted acrylamides with acrylic acids and substituted acrylic acids.

### 21. Penetration Enhancers

Penetration enhancers, which can be included in the cosmetic skin formulations, are substances that facilitate the absorption of penetrant through the skin membranes by temporarily diminishing the impermeability of the skin. Ideally, these materials should be pharmacologically inert, nontoxic, nonirritating, non-allergenic, odorless, tasteless, colorless, and have good solvent properties. The enhancer should not lead to the significant loss of body fluids, electrolytes, and other endogenous materials, and skin should regain its barrier properties on its removal within an acceptable period of time. No single penetration enhancer can possess all the required properties. However, many enhancers exhibit many of these attributes, and they have been described (for example as reviewed in Drug Development and Industrial Pharmacy 2000, 26, 1131-1140).

### 22. Anti-Histamines

The cosmetic skin formulations optionally include anti-histamines, also called histamine antagonists, which are substances that inhibit the action of histamine by blocking it from attaching to histamine receptors; or by inhibiting the enzymatic activity of histidine decarboxylase, catalyzing the transformation of histidine into histamine; or similar. Examples of suitable anti-histamines are acrivastine, azelastine, brompheniramine, buclizine, bromodiphenhydramine, carbinoxamine, cetirizine, chlorpromazine, cyclizine, chlorpheniramine, chlorodiphenhydramine, cimetidine, clemastine, cyproheptadine, desloratadine, dexbrom-pheniramine, deschlorpheniramine, dexchlorpheniramine, dimenhydrinate, dimetindene, diphenhydramine, doxylamine, ebastine, embramine, famotidine, fexofenadine, lafutidine, levocetirizine, loratadine, meclozine, mirtazapine, nizatidine, olopatadine, orphenadrine, phenindamine, pheniramine, phenyltoloxamine, promethazine, pyrilamine, quetiapine, ranitidine, roxatidine, rupatadine, tripelennamine, and triprolidine.

An anti-histamine may be included in the cosmetic skin formulations described herein. In one instance, an anti-histamine is included at a level of from about 0.01% to about 10%, or from about 0.5% to about 5%, by weight of the formulation. The exact content (%) of anti-histamine to be used in the formulations depends on the particular anti-inflammatory agent utilized since such agents can vary in potency.

### 23. Preservatives

The cosmetic skin formulations optionally include one or more preservatives in the skin formulations to prevent microbial growth in the formulations. Suitable preservatives include, but are not limited to, glycerin containing compounds (e.g., glycerin or ethylhexylglycerin or phenoxyethanol), 1,2-hexanediol, propanediol, benzyl alcohol, parabens (methylparaben, ethylparaben, propylparaben, butylparaben, isobutylparaben, etc.), sodium benzoate, ethylenediamine-tetraacetic acid (EDTA), potassium sorbate, and/or grapefruit seed extract, or combinations thereof. More than one preservative may be included in the formulation. Other preservatives are known in the cosmetics industries and include sodium chloride, salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol).

A preservative may be added to the cosmetic skin formulations described. In one instance, a preservative is added at a level of from about 0.01% to about 10%, or from about 0.5% to about 5%, by weight of the formulation. The exact content (%) of preservative to be used in the formulations depends on the particular preservative utilized since such agents can vary in potency. In some instances, the preservative(s) are optionally included in an amount ranging from about 0.1% to about 5% by weight of the formulation, preferably from about 0.3% to about 3% by weight of the formulation. Preferably, the formulations are paraben free.

### 24. Ceramides

The cosmetic skin formulations optionally include one or more ceramides. Suitable ceramides include, but are not limited to, ceramide AP, ceramide EOP, ceramide NS, ceramide NP, ceramide NG, and combinations thereof. In one instance, a ceramide is added at a level of from about 0.01% to about 10%, or from about 0.5% to about 5%, by weight of the formulation. The exact content (%) of ceramide to be used in the formulations depends on the particular ceramide utilized since such agents can vary in potency.

### 25. Exfoliants

The cosmetic skin formulations optionally include one or more a ceramide selected from, but not limited to, alpha hydroxy acids, for example lactic acid, glycolic acid, mandelic acid, tartaric acid and malic acid, to facilitate the removal of dead cells and stimulate the formation of collagen and elastin. In some instances, an exfoliant is added at a level of from about 0.01% to about 10%, or from about 0.5% to about 5%, by weight of the formulation. The exact content (%) of exfoliant to be used in the formulations depends on the particular exfoliant utilized since such agents can vary in potency.

The skin formulations optionally include one or more additional substances (excipients, carriers, additives), such as those described herein, which should provide some benefit to the object of the formulation. In some instances, the selected additional substances may lead to an enhanced efficacy of the formulation, as compared to the formulation absent the additional substances. The enhanced efficacy can be additive (the sum of efficacies of the individual agents alone), or more than additive (larger than the sum of efficacies of the individual agents alone). A decision to include an additional ingredient or substance and the choice of a specific ingredient or substance depends on the specific use of the formulation.

d. Forms of Cosmetic Skin Formulations

The cosmetic skin formulations described may be in any suitable physical form. Suitable forms can include, but are not limited to, liquids, lotions, milks, gels, emulsions (i.e, macro-emulsions, micro-emulsions, nano-emulsions, oil-in-water, silicone-in-water, water-in-oil, water-in-silicone, water-in-oil-in-water, oil-in-water-in-oil, and oil-in-water-in-silicone emulsions), balms, creams, serums, balms, colloids, solutions, suspensions, dispersions, pastes, ointments, oils, and the like. In some instances, the cosmetic skin formulations can be present on at least a part of patch, a microneedle device, or form at least part of any other cosmetic, or dermatologically acceptable formulation or device. In some instances, the cosmetic skin formulations are a skin rejuvenation product, a skin conditioner, a skin moisturizer. In some instances, the cosmetic skin formulations are present in or form part of a skin patch (i.e., iontophoretic patches, microprojection patches, microneedle patches), a skin mask, a tissue wipe, a foundation, a lip liner, a lip gloss, a lip balm, a sunscreens, a sunblock, a soap, a body scrub, an exfoliant (such as malic acid), a cold cream, a cleanser, a rinse, and/or any other forms readily known to those skilled in the art.

In some particular instances, the cosmetic skin formulation is a serum, such as a face serum. In some other particular instances, the cosmetic skin formulation is a skin moisturizer formulation. In still other instances, the cosmetic skin formulation is a scalp formulation, such as a scalp serum.

The cosmetic skin formulations described can have any suitable viscosity depending on the form of the formulation. In some instances, the viscosities of can range from about 100 cps to about 1,000,000 cps, from about 100 cps to about 500,000 cps, from about 100 cps to about 100,000 cps, from about 100 cps to about 75,000 cps, or from about 1000 cps to about 50,000 cps, when measured by Brookfield DV-II pro Viscometer with low viscosity spindle No. 4 at 20 rpm, as well as sub-ranges and individual viscosity values contained within the aforementioned ranges.

### III. Kits

The cosmetic skin formulations described herein may be provided as part of a cosmetic kit. The kits may optionally include instructions for use of the kit.

The kit may further contain one or more different cosmetic skin formulations, which may have different purposes, such as to provide different effects and/or improvements in appearance of skin. The kit may optionally contain one or more other skin formulations which are commercially available and which do not fall within the scope of the cosmetic skin formulations described herein. Each of the cosmetic skin formulation(s) and optional skin formulation(s) are typically provided in separate suitable containers and/or compartments forming the kit.

In some instances, a kit may include at least one cosmetic skin formulation, which is in the form of a serum, and at least one other cosmetic skin formulation which is a moisturizer. Such a kit may further include one or more other skin formulations, such as a skin cleanser. In certain instances, the serum is a face serum. In some other instances, the kit may also include a scalp formulation, optionally in the form of a serum.

In one instance, a kit includes at least one cosmetic skin formulation in the form of a serum. In another instance, a kit includes one cosmetic skin formulation in the form of a serum and one cosmetic skin formulation in the form of a moisturizer. In yet another instance, a kit includes one cosmetic skin formulation in the form of a serum, one cosmetic skin formulation in the form of a moisturizer, and another skin formulation that is a skin cleanser.

### IV. Methods of Use

The cosmetic skin formulations described herein are used to treat skin. In one non-limiting instant, a method for treating skin includes:
(1) applying a cosmetic skin formulation described to the skin of a subject.

The subject is typically a mammal, such as a human.

In some instances, the cosmetic skin formulation is applied topically onto any areas of the face, neck, neckline, decollete, scalp, hand, palm, arm, leg, foot, sole, chest, breast, back, abdomen, buttock, and/or any other skin areas of the subject. In some instances, the cosmetic skin formulation is applied to the face. In some particular instances, the cosmetic skin formulation is in a form suitable for application to the skin of a subject's face and/or scalp and the formulation is optionally in the form of a serum.

In some instances, the cosmetic skin formulation, once applied, remains on the skin for a period of up to 24 hours, up to 20 hours, up to 15 hours, up to 10 hours, up to 8 hours, up to 7 hours, up to 6 hours, up to 5 hours, up to 4 hours, up to 3 hours, up to 2 hours, or up to 1 hour.

In some other instances, the skin formulation remains on the skin for about 1 minute to about 30 minutes, about 1 minute to about 20 minutes, about 1 minute to about 15 minutes, about 1 minute to about 10 minutes, about 5 minutes to about 30 minutes, about 5 minutes to about 20 minutes, about 5 minutes to about 15 minutes, or about 5 minutes to about 10 minutes.

In still other instances, the cosmetic skin formulation can be optionally removed from the skin following step (1), such as by rinsing the skin with water or cleansing the skin with a cleanser and water. In certain other instances, there is no need to remove the formulation from the skin once applied because the formulation is fully absorbed by the skin.

In some instances, the cosmetic skin formulation can be also applied in conjunction (i.e., before, after, or simultaneously) with the use of a skin device(s) for changing the penetration characteristics of skin, such as, for example, laser, light, infrared, radiofrequency, ultrasound, electroporation, sonophoresis, thermal, plasma, and/or high pressure devices, and/or any combination(s) (including combination devices) thereof. Any other commonly used means of administration can also be utilized.

In some instances, the cosmetic skin formulation is supplied with usage/application instructions. In one non-limiting example, instructions may include the following guidance, for example: apply an amount of the skin formulation product onto the skin, such as of the face and/or body following use of a cleanser and/or toner, at least once a day (morning or night) or twice a day (morning and night) prior to applying any other skin care formulations and/or following a typical skin regimen used by the user.

In some instances, the cosmetic skin formulation is a scalp formulation which is applied, such as in the form of a serum, on a daily basis. The scalp formulation can be applied to dry or damp hair and massaged into the scalp to distribute the formulation and absorbance thereof into the scalp. In some instances, the scalp formulation is applied to natural part lines and/or along hairlines of a subject.

a. Evaluating Efficacy of Cosmetic Skin Formulations

Methods and processes for testing and evaluating the efficacy of a cosmetic skin formulation are known. For instance, maintaining skin, the prevention, amelioration, and/or treating of the signs of skin aging, protection and/or improving of a skin condition, and the prevention and/or treatment of skin imperfections are functional features which can be visualized, analyzed, measured and quantified using many techniques known by the specialist in cosmetic skin treatments.

In some instances, the skin formulations described can be evaluated for their ability to decrease fine lines, wrinkles, skin folds, and skin roughness, which can be quantified either directly on the person contact-free using fringe projection (FOITS=Fast Optical In vivo Topometry System; DERMATOP™ or PRIMOS™ system), or by silicon replicas of the skin area which are then analyzed by the technique called “drop shadows” or by a FOITS system, or by a Canfield VISIA™ device. Changes in volume and shape of the face can be quantified using a relief obtaining system without contact using a fringe projection FOITS system. Alteration of the skin barrier can be quantified by measuring transepidermal water loss (TEWL) using a TEWAMETER™, a VAPOMETER™, a DERMALAB™, and/or an AQUAFLUX™ device. Loss of firmness and/or elasticity and/or tone and fatigue of the skin can be quantified using a CUTOMETER™, a REVISCOMETER™, an AEROFLEXMETER™, a DYNASKIN™, a BALLISTOMETER™, a TWISTOMETER™ and/or a DERMALAB™ device. Dull complexion, loss of uniformity of skin tone, pigmentation changes (hypo and hyper pigmentation), local reddening, loss of clarity and brightness of the complexion, pigmentation spots, rosacea, dark circles are directly measurable using a MEXAMETER™, a CHROMAMETER™, a COLORMETER™, a Canfield VISIA™, a Canfield VISIA-CR™, a SIASCOPE™, a GONIOLUX™ or a confocal laser microscope device, and/or by specific color analysis on photo (enabled by the technique of photographing in polarized crossed and parallel light). The number and size of facial pores can be quantified by the silicon replica technology described above, or by specific analysis on photo (enabled by using a video microscope or a macroscopic photographing system). Atrophy and thinning of the skin, epidermis, dermis, or hypodermis (e.g., in case of studying slimming agents) is measurable by measuring TEWL (e.g., in case of studying the epidermis), or by an ultrasound echographic device, and/or a confocal laser microscope device. Density of skin fibers can be quantified by ultrasound and then by image analysis. Cellulite is quantified either directly by a relief obtaining system without contact using fringe projection (FOITS) or indirectly by measuring the length of the dermo-hypodermal junction by an ultrasound echographic device. Stretch marks are either directly quantified using a relief obtaining system without contact using fringe projection (FOITS) or by the silicon replica technology. Skin softness is directly measurable by techniques of friction study as with a frictiometer device or indirectly by the silicon replica technology. Changes in collagen, extracellular matrix components, and/or in connective tissue fibers may be quantified by histology, confocal laser microscopy, UV spectroscopy, SIASCOPE™, and/or by multiphoton spectroscopy. All changes visible to the eye (including but not limited to fine lines, wrinkles, folds, texture, sagging, loss of elasticity color, tone, pigmentation, redness) can be quantified in direct or on photography, by a trained judge person (or panel of judges) or not, with or without visual scoring system (e.g., using a 4-point severity scale).

Some of the benefits and effects associated with use of the skin formulations described herein may be evaluated by a trained individual or a panel of trained individuals) using a scoring system or scale. By comparing the scores on a skin condition (such as fine lines, wrinkles, folds, texture, sagging, loss of elasticity color, tone, pigmentation, redness, etc.) before and after treatment with a cosmetic skin formulation, it is possible to determine an increase/decrease in the score due to application of the cosmetic skin formulation. By comparing the scores on a skin condition (such as fine lines, wrinkles, folds, texture, sagging, loss of elasticity color, tone, pigmentation, redness, etc.) at two different times between application of the cosmetic skin formulation (such as, for example, after week two and week five following once or twice daily application of the formulation), it is possible to determine an increase/decrease in the score due to application of the cosmetic skin formulation over time. In some instances, where a higher score represents an improvement in a skin property or condition, the score following treatment with the cosmetic skin formulation is increased by at least one point or higher. In some instances, where a lower score represents an improvement in a skin property or condition, the score following treatment with the cosmetic skin formulation is decreased by at least one point or more. In some instances, where a higher score represents an improvement, the score following treatment with the cosmetic skin formulation is increased by at least about 10%, 20%, 30%, 40%, 50%, 60%, 70%, 80%, 90%, or 100%, or higher, compared to the score before treatment with the cosmetic skin formulation. In some instances, where a lower score represents an improvement, the score following treatment with the cosmetic skin formulation is decreased by at least about 10%, 20%, 30%, 40%, 50%, 60%, 70% A, 80%, 90%, or 100%, or higher, compared to the score before treatment with the cosmetic skin formulation.

In some instances, an effect on one or more properties of the skin is evaluated on an ordinal scale of a given number of points, such as a 0-5 scale (where 0=no effect, 1=minimal effect, 2=mild effect, 3=moderate effect, 4=high effect), the effect is evaluated by looking at changes in the score between two periods of time (such as prior to and following a first treatment, or between two periods of time, such as the period of time ranging from prior to a first treatment and after one week, after two weeks, after three weeks, after four weeks, after two months, after three months, after four months, after five months, after six months, after seven months, after eight months, after nine months, after ten months, after eleven months, or after a year of treatments). For example, an ordinal score may be assigned to a skin feature(s), including but not limited to facial erythema, fine lines, wrinkles, radiance/brightness, skin roughness (tactile), skin roughness (visual), and/or overall appearance, before applying a skin formulation and compared to the score at a second (subsequent) time following one or more treatments with the skin formulation. In some instances, comparison is between the scores between two times both following the application of the formulation to skin, such as when repeated applications occur over time. The score can change between two given times during treatment with the cosmetic skin formulation, such as between week one and week four. In some instances, the score increases between two given time periods, where an increase is considered desirable. In some instances, the score decreases between the two given time periods, where a decrease is considered desirable. In some instances, the score increases or decreases by at least one point, as may be possible on the ordinal scale chosen, representing an improvement in a desired effect (such as on radiance/brightness; overall appearance) and/or an alleviation or amelioration in an undesired effect (such as on facial erythema, wrinkles, skin roughness) on the skin by use of the skin formulation.

In some instances, the skin formulations provide an effect on one or more properties of the skin, as compared to untreated skin or a control skincare formulation. The effect may be an improvement in one or more desired skin properties and/or a reduction/decrease in one or more undesirable skin properties. For example, the one or more properties can include, without limitation, improvement to skin texture, improvement to skin bounce-back on pinching or pressing the skin, improvement to skin firmness, reduction to skin redness, reduction to skin inflammation, increase in smoothness of lines and/or wrinkles, decrease in fine lines and/or wrinkles, reduction in visibility and/or prominence of fine lines and/or wrinkles, improvement in smoothness of skin, improvement in softness of skin, improvement in hydration of skin, improvement in plumpness of skin, reduction in dryness of skin, reduction in flakiness of skin, improvement in radiance of skin, improvement in luminosity of skin, improvement in brightness of skin, improvement in appearance of skin, or improvement in glow of skin, or any combination thereof. The improvement in the one or more properties of the skin can be evaluated using the various methods known in the art, including those described herein.

In some instances, the skin formulation is a serum; wherein barrier function, brilliance, hydration, smoothness, firmness, elasticity, texture, and/or luminosity of the skin is improved after a period of time, as compared to the skin prior to application of the serum to the skin of a subject; and/or wherein redness, lines, and/or wrinkles of the skin reduced after a period of time, as compared to the skin prior to application of the serum to the skin of a subject. Such improvements or reductions can be evaluated by, for example, visual and/or tactile grading, or other suitable methods described herein, following the period of time, as compared to the skin prior to application of the serum to the skin of a subject.

### 1. Gene Expression Analysis

In some instances, the skin formulations can be evaluated for their ability to up-regulate or down-regulate expression of one or more genes following one or more applications to the skin.

Gene expression can be assessed using standard methods that are known in the art, and can include determining target mRNA levels in sample of cells. RNA can be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, reverse transcriptase PCR (RT-PCR), quantitative PCR analysis, RNase protection assays, Northern blotting, in situ hybridization, serial analysis of gene expression (SAGE), differential or subtractive hybridization, microarrays, and sequencing techniques such as next generation sequencing (NGS) and/or RNA-Seq. The analysis can be of one, two, three, four, five, or more individual targets, and/or an analysis of the whole transcriptome. Transcriptome refers to the set of all RNA coding transcripts and optionally all non-coding transcripts in a single cell or a population of cells.

Gene expression analysis can be comparative analysis of gene expression under two or more different conditions; and/or among two of more different cell types; etc. For example, in some forms, gene expression of one or more targets, or the whole transcriptome, before treatment with the skin formulation is compared to gene expression of one or more targets, or the whole transcriptome, after one or more treatments with the skin formulation. In some forms, gene expression of one or more targets, or the whole transcriptome, after one or more treatments with the skin formulation is compared to gene expression of one or more targets, or the whole transcriptome, after one or more additional, subsequent treatments with the skin formulation.

For instance, the skin formulations can be evaluated in vitro for a gene-level view of changes in the human transcriptome from cultured human 3D skin tissue model, such as EPIDERM-FT™. On this basis, differential gene expression can be quantified using a microarray method, such as a CLARIOM™ S microarray method. Such testing methods allow for global gene expression profiling from 21,448 well-annotated human genes which can be analyzed by Affymetrix Human CLARIOM™ S arrays, where significant expression changes can be filtered/identified using fold change threshold of: <−2 or >2 versus a placebo or versus no treatment. Typically, the genes evaluated affect or regulate (i.e., up- or down-regulate) reinforcement of the skin barrier function, cornified envelope development, and/or cornified envelope lipid synthesis. Examples of such gene expression testing are detailed below and in the Examples section.

In some instances, when tested for gene expression, the skin formulation up-regulates or down-regulates one or more, and optionally all, of the following genes: involucrin (IVL), keratin 1 (KRT1), transglutaminase 1 (TGM1), filaggrin (FLG), loricrin (LOR), and combinations thereof.

In some instances, when tested for gene expression, the skin formulation up-regulates or down-regulates one or more, and optionally all, of the following genes: TNF-a, interleukin-6, and interleukin 8.

In still other instances, when tested for gene expression, the skin formulation up-regulates one or more, and optionally all, of the following genes: involucrin (IVL), keratin 1 (KRT1), transglutaminase 1 (TGM1), filaggrin (FLG), loricrin (LOR), and combinations thereof, and the skin formulation down-regulates one or more, and optionally all, of the following genes: TNF-a, interleukin-6, and interleukin 8 and optionally nuclear factor of kappa light polypeptide gene enhancer in B-cells 1 (NFKB1).

In some instances, the skin formulation regulates (i.e., up- or down-regulates) genes associated with reinforcement of the skin barrier function include one or more of ceramide synthase 1 (CERS1), ceramide synthase 2 (CERS2), ceramide synthase 3 (CERS3), ceramide synthase 4 (CERS4), ceramide synthase 5 (CERS5), ceramide synthase 6 (CERS6), tight junction protein 1 (TJP1), occludin (OCLN), filaggrin (FLG), prostaglandin-endoperoxide synthase 2 (PTGS2), and nuclear factor of kappa light polypeptide gene enhancer in B-cells 1 (NFKB1), involucrin (IVL), keratin 1 (KRT1), transglutaminase 1 (TGM1), loricrin (LOR), and combinations thereof.

In other instances, when tested for gene expression, the skin formulation up- or down-regulates one or more of the following genes cornified envelope development: loricrin (LOR), small proline-rich protein 1A (SPRR1A), small proline-rich protein 1B (SPRR1B), small proline-rich protein 2A (SPRR2A), small proline-rich protein 2B (SPRR2B), small proline-rich protein 2D (SPRR2D), small proline-rich protein 2E (SPRR2E), small proline-rich protein 2F (SPRR2F), small proline-rich protein 2G (SPRR2G), small proline-rich protein 3 (SPRR3), and small proline-rich protein 4 (SPRR4), and combinations thereof.

In still other instances, when tested for gene expression, the skin formulation up- or down-regulates one or more of the following genes that regulate cornified envelope lipid synthesis: UDP-glucose ceramide glucosyltransferase (UGCG), fatty acid synthase (FASN), late cornified envelope 1A (LCE1A), late cornified envelope 1B (LCE1B), late cornified envelope 1C (LCE1C), late cornified envelope 1D (LCE1D), late cornified envelope 1E (LCE1E), late cornified envelope 1F (LCE1F), late cornified envelope 2A (LCE2A), late cornified envelope 2B (LCE2B), late cornified envelope 2C (LCE2C), late cornified envelope 2D (LCE2D), late cornified envelope 3A (LCE3A), late cornified envelope 3B (LCE3B), late cornified envelope 3C (LCE3C), late cornified envelope 3D (LCE3D), late cornified envelope 3E (LCE3E), late cornified envelope 4A (LCE4A), late cornified envelope 5A (LCE5A), and late cornified envelope 6A (LCE6A), and combinations thereof.

### 2. Clinical Study of Skin Formulations

The skin formulation can be evaluated using a clinical study. In one non-limiting example, a clinical study can rely on any combination of expert visual/tactile grading, corneometer measurements, TEWL measurements, Cutometer measurements, vivaScope measurements, SkiCon measurements, and Self-Perception Questionnaires when studying a group of subjects using the skin formulation once or twice daily over a period of time, such as over about 3 months or 90 days of use at home, when following the directions of usage provided to the study participants.

The efficacy of the skin formulation for improving skin elasticity and firmness can be assessed by a suction method, such as by using a Cutometer®. The efficacy of the formulation for keeping skin in a good condition, or skin conditioning efficacy, can be assessed by transepidermal water loss (TEWL) measurements, such as by using a Tewameter®.

Exemplary parameters for a clinical study are provided below. However, clinical studies can be varied and designed in any suitable manner in the field of skincare.

### Exemplary Study Methods:

A washout product can be used by the study subjects prior to the first application of the skin formulation being evaluated.

The skin formulation is supplied to the subjects with instructions to follow usage instructions. Exemplary usage directions are:

Apply a generous amount of formulation on face and forearm, one to two pumps, after cleansing/toner, twice a day (morning and night) prior to applying the study participant's regular regimen. No other serums/moisturizers should be used. For each day, each study participate should indicate whether he/she used the product.

### Exemplary Study Procedure:

### Visit 1—Washout (Day-5):

Subjects are provided with a washout product, to replace their usual facial cleansers and to be used daily for 5 continuous days on the face and forearms prior to study visit 2. Subjects use a usage diary to track the washout progress.

Subjects meeting enrollment criteria were provided with a washout product, to replace their usual facial cleansers and to be used daily for 5 continuous days on the face and forearms prior to study visit 2. A usage diary was issued to subjects to track the washout progress. Instructions were given to subjects not to apply any creams or moisturizers to the face or forearms starting from 2 days prior to visit 2 and, for the entire duration of the study. Subjects were also instructed to come to all study visits without makeup. Subjects also brought along their diary and washout product to the next visit and washed their face 2.5 hours prior to their visit.

### Visit 2—Baseline (Study Day 1):

Subjects attend a testing facility where eligibility is verified, and diaries checked, and washout product collected. Once accepted into the study, subjects are acclimatized to indoor ambient conditions, temperature 20-22° C. and relative humidity 30-50%, for at least 30 minutes before assessments. Baseline measurements are then taken, where the subject's face is assessed visually and tactilely by an expert grader. Corneometer, SkiCon and TEWL assessments are also assessed using the forearm, while Vivascope and Cutometer assessments are conducted on the face.

Once baseline assessments are completed, the skin formulation is applied to the face and the forearm after which expert visual assessments of the face and Corneometer, SkiCon and TEWL assessments of the forearm are reassessed.

Subjects also complete a Self-perception Questionnaire (SPQ). Subjects are then issued with the skin formulation and usage instructions to take home and use as instructed for the next 3 months. Subjects also ensured that their face was washed no less than 2.5 hours prior to each visit.

### Visit 3—Study Day 7:

Subjects return to the testing facility after using the product (as instructed) at home for 7 days. Test articles are weighed, and diaries are checked for compliance. Subjects also acclimatised to indoor ambient conditions, temperature 20-22° C. and relative humidity 30-50%, for at least 30 minutes before assessments. The subject's face is then visually and tactilely assessed by an expert grader, and Corneometer, SkiCon and TEWL assessments are assessed using the forearm and Cutometer assessments are conducted on the face. Subjects also complete a self-perception questionnaire.

### Visit 4—Study Day 30:

Subjects return to the testing facility after using the product (as instructed) at home for 30 days. Test articles are weighed, and diaries are checked for compliance. Subjects also acclimatised to indoor ambient conditions, temperature 20-22° C. and relative humidity 30-50%, for at least 30 minutes before assessments. The subject's face is visually and tactilely assessed by an expert grader, and Corneometer, SkiCon and TEWL assessments are assessed using the forearm. Subjects also complete a self-perception questionnaire.

### Visit 5—Study Day 60:

Subjects return to the testing facility after using the product (as instructed) at home for 60 days. Test articles are weighed, and diaries are checked for compliance. Subjects also acclimatised to indoor ambient conditions, temperature 20-22° C. and relative humidity 30-50%, for at least 30 minutes before assessments. The subject's face is visually and tactilely assessed by an expert grader, and Corneometer, SkiCon and TEWL assessments are assessed using the forearm and VivaScope assessments were conducted on the face. Subjects also complete a self-perception questionnaire.

### Visit 6—Study Day 90 (Final Visit):

Subjects return to the testing facility after using the product (as instructed) at home for 90 days.

The same measurements were taken and inspections were conducted as in Visit 5.

Upon completion of all assessments and receipt of all diaries and test articles, each subject's participation is considered complete.

### Exemplary Visual Assessments:

### Expert Visual and Tactile Grading:

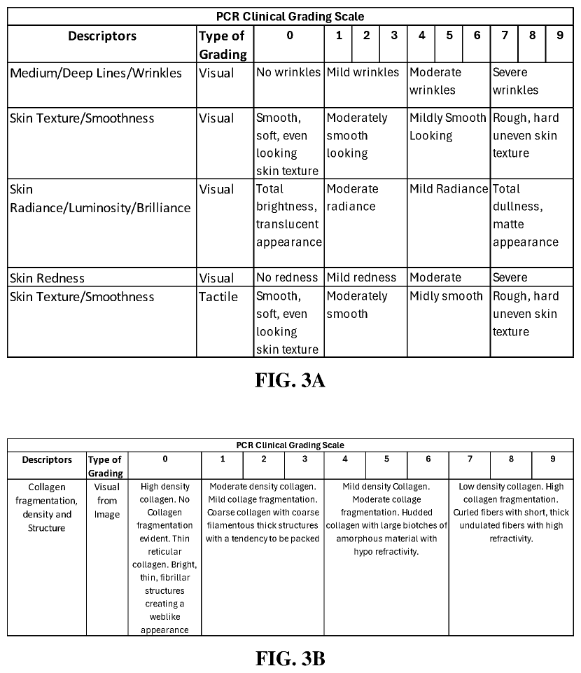

Visual assessment is conducted on the face at baseline, after immediate use (1 hour), Day-7, Day-30, Day-60, and Day-90, while tactile assessment is conducted on the face at baseline, Day-7, Day-30, Day-60, and Day-90. The same Expert grader assesses all subjects for the duration of the study according to the grading scales below. Illumination of the subject's face was by a 60-watt pearl bulb, approximately 30 cm from the site. The PCR Clinical Grading Scale, which can be used in a study, is shown in FIG. 3A.

### Instrumental Assessments:

### Corneometer

Moisturization measurements are performed using the Corneometer CM825 (Courage and Khazaka, Germany). This instrument relies on the dielectric constant, a physical property of water, which is relatively high and as such will affect the capacitance of a capacitor. Any change in the dielectric constant due to skin moisture variations will alter the capacitance of the precision capacitor in the instrument. These variations are detected electronically and are converted into a value by the Corneometer.

Triplicate readings are made using the probe attachment of the Corneometer at the test site on the forearm, between each assessment, the probe attachment of the Corneometer is pressed onto a dry tissue. Subjects are in a controlled environment (at a temperature of 20-22° C. and at a relative humidity of 30-50%) for at least 15 minutes prior to any assessment being performed. If one measurement is more than ±10 units (outlier) from the other measurements, a fourth measurement is taken.

Measurements are taken at baseline, after immediate use (1 hour), Day-7, Day-30, Day-60, and Day-90.

### SkiCon

Skin conductance is an alternative approach for measurement of skin moisture derived from alterations in the electrical properties of the stratum corneum (SC). This device can overcome the issue of false or inaccurate readings experienced by capacitance or impedance measurements due to external influences. SkiCon can reduce these influences by applying a higher frequency of 3.5 MHz on closely spaced electrodes inside a probe that maintains the electric field within the superficial layers of the skin.

SkiCon 200EX (IBS co. Hamamatsu, Japan) measures skin conductance at a fixed high frequency current of 3.5 MHz. The apparatus gives readings of conductance in siemens (μS) units, ranging from 0-1999. All conductance measurements are given after a reading of 3 s.

Measurements are taken from the forearm at baseline, after immediate use (1 hour), Day-7, Day-30, Day-60, and Day-90.

Trans epidermal water loss (TEWL) by Tewameter® Probe

Evaporation of water from the skin occurs normally as part of the skin metabolism. However, when barrier function of the skin becomes even slightly damaged, water loss will increase. Trans epidermal water loss can be measured with a Tewameter® TM300 Probe (Courage and Khazaka; Koln, Germany). This method is an effective method to measure barrier function of the skin. The measurement of the water evaporation, and therefore TEWL, is based on the diffusion principle in an open chamber and is measured as g/m²/h. The density gradient is measured indirectly by two pairs of sensors in the probe attachment, one for temperature and the other for relative humidity. This density gradient is then analysed by a microprocessor in the instrument.

Measurements are taken from the forearm at baseline, after immediate use (1 hour), Day-7, Day-30, Day-60, and Day-90.

### VIVASCOPE® 1500—Assessment of Collagen:

The structure and appearance of collagen using confocal laser scanning microscopy, determined by the relationship between reticulated, coarse, huddled, and curled collagen fibers, changes with age. Younger-looking healthy skin predominantly shows thin reticulated fibers. As a person ages, the proportion of thin reticulated fibers progressively decreases in favor of coarse, huddled and curled collagen fibers in older people. These structural changes are recognizable in confocal images. As the skin ages, the dermal collagen network becomes increasingly fragmented, which is characterized by shorter, less organized fibers that accumulate as degraded collagen fragments. The younger and healthier the skin, the larger, more diffuse, and less fragmented the collagen structures are.

For Vivascope assessment, subjects are in a controlled environment (at a temperature of between 20° C.-24° C. and relative humidity of 35%-50%) for 30 minutes prior to any assessments being performed. Measurements are taken from the right crow's feet area on the outer corner of the eye at baseline, Day-60, and Day-90.

The test area is captured with a VivaCam, which allows the confocal and photographic images to be correlated.

The collagen structure (i.e., the fragmentation and density of collagen in the papillary and superficial dermis) is visualized in vivo by confocal laser scanning microscopy (VIVASCOPE® 1500; VivaScope GmbH, Munich, Germany). This allows the cellular microstructure to be visualized as optical cross sections with a horizontal resolution of up to 1.25 μm and vertical resolution of 3-5 μm, up to a maximum depth of the superficial dermis (200 μM). Collagen fibers appear as white structures.

Individual images (500 μm×500 μm) are taken (approx. 40) at 5 μm scanning steps to create a stack of image. Only the images targeting the collagen network (in the papillary and superficial dermis) are analysed. Images taken at baseline are compared to those taken at Day-60 and separately to those at Day-90. The images for each are graded for collagen fragmentation and density by an expert grader on a visual analog scale The PCR Clinical Grading Scale used for Vivascope assessment of collagen is shown in FIG. 3B.

### Firmness (R0)/Elasticity (R2) by Cutometer®:

The viscoelastic properties of the skin (elasticity and firmness) can be measured using a Cutometer® MPA 580 (Courage and Khazaka, Germany) at baseline, Day-7, and Day-90. The measuring principle is based on the suction method. Negative pressure is created in the device and the skin is drawn into the aperture of the probe.

Inside the probe, the penetration depth is determined by a non-contact optical measuring system. This optical measuring system includes a light source and a light receptor, as well as two prisms facing each other, which project the light from the transmitter to receptor. The light intensity varies due to the penetration depth of the skin. The measurements for passive stretching are recorded. This measurement can be displayed as curves at the end of each measurement using Windows® based software. Measuring mode 1 is used with constant suction of 400 mbar for five seconds followed by relaxation time of 3 seconds with three repetitions. Elasticity parameters measured are R2 and R0, which serves an indication of firmness, and an average of the repetitions (e.g., 3 repetitions) are reported.

The following R-Parameters can be calculated using the Cutometer® MPA 580:
R0: This parameter represents the passive behavior of the skin to force.
R1: The ability of the skin to return to its original state.
R2: Gross elasticity, the closer the value is to 1 (100%), the more elastic the curve, very important parameter.
R3: Maximum amplitude of the last suction curve after repeated suction. “Tiring effects” of the skin are visible, as the amplitude increases with each new suction.
R4: Last minimum amplitude compared to the first curve, “tiring effects” of the skin are visible, as the ability of re-deformation decreases with each new suction.
R5: Net elasticity, the higher the value, the more elastic is the skin.
R6: Portion of the visco-elasticity on the elastic part of the curve. The smaller the value the higher the elasticity.
R7: Portion of the elasticity compared to the complete curve, the closer the value is to 1 (100%), the more elastic the skin.
R8: Skin recovery, the closer the value is to R0, the better is the ability of the skin to return to its original state.
R9: Represents tiring effects of the skin after repeated suction and release of the skin. The smaller R9, the smaller the tiring effects.

The Cutometer can be used to calculate other skin parameters, including F-Parameters and Q-Parameters. F-Parameters relate to area parameters that can only be taken in mode 1, where some need 10 repetitions. The Q-Parameters can be measured to evaluate correlations between skin age and the viscoelastic recovery of the skin.

### Self-Perception Questionnaire:

Study subjects can be instructed to complete a self-perception questionnaire on multiple days of treatment, such as: Day-1 (Immediately after use, up to 1 hour), Day-7, Day-30, Day-60, and Day-90.

b. Effects of the Skin Formulations on the Skin

The methods of treating skin using the formulations described above can provide a skin rejuvenation effect and/or an improvement in the appearance of the skin, as measured via visual inspection, following step (1), as compared to the skin prior to step (1).

In some instances, the methods provide an effect of maintaining healthy skin; restoring damaged skin, enhancing the restoration of skin after cosmetic and dermatological procedures; enhancing wound healing; or combinations thereof.

In still other instances, the methods can treat, alleviate, or ameliorate one or more visible signs of aging of the skin, where the signs of aging can include, but are not limited to, fine lines, wrinkles, enlarged pores, roughness, dryness, loss of elasticity, loss of volume, atrophic skin, stretch marks, puffy eyes, dark (under eye) circles, and combinations thereof.

Treating, alleviating, or ameliorating signs of aging of the skin can include delaying, minimizing and/or preventing visible and/or tactile discontinuities in skin (e.g., texture irregularities in the skin which may be detected visually or by feel) or ameliorating, e.g., diminishing, minimizing and/or effacing, discontinuities in skin.

Certain benefits of use of the cosmetic skin formulations according to the methods described (e.g., moisturization, soothing, calming, tightening, smooth feel, etc.) can be noticed within a few hours (i.e., about 1, 2, 3, 4, 5, 6, 7, 8, 9, 10, 11, 12, 13, 14, 15, 16, 17, 18, 19, 20, 21, 22, or 23 hours) to a few days (i.e., about 1, 2, 3, 4, 5, 6, 7 days) after topically applying the formulations according to the methods. In some instances, it can take weeks (i.e., about one, two, three, or four weeks) or at least a few months (i.e., about one, two, three, four, five, or six months) to notice benefits (e.g., anti-aging, wrinkle reducing, skin lightening, anti-redness, improving atrophy, wound healing, etc.). In certain instances, the skin formulation is applied to skin at least once to twice a day. However, determination of an effective dose of any of the formulations to achieve a desired effect(s) is within the routine level of skill in the art.

For the aforementioned effects, these can be evaluated using suitable techniques/methods, such as those described above, and/or generally known in the field of skincare.

In some instances, the method improves radiance, brilliance, and/or luminosity of the skin, as compared to the skin prior to step (1′), by visual grading. In some instances, the radiance, brilliance, and/or luminosity of the skin is improved by at least about 1%, 5%, 10%, 15%, 20%, 25%, 30%, 35%, 40%, 45%, 50%, 55%, 60%, 65%, 70%, 75%, 80%, 85%, 90%, 95%, or 100%; or wherein the radiance, brilliance, and/or luminosity of the skin is improved by an amount ranging from about 1% to 100%. In some other instances, the radiance, brilliance, and/or luminosity of the skin is improved by at least about 75%, 80%, 85%, 90%, 95%, or 100%; or wherein the radiance, brilliance, and/or luminosity of the skin is improved by an amount ranging from about 75% to 100%. In still other instances, the radiance, brilliance, and/or luminosity of the skin is improved by at least about 90%, 95%, or 100%; or wherein the radiance, brilliance, and/or luminosity of the skin is improved by an amount ranging from about 90% to 100%.

In some instances, the method reduces or eliminates redness of the skin, as compared to the skin prior to step (1′), by visual grading. In some instances, the redness of the skin is reduced by at least about 1%, 5%, 10%, 15%, 20%, 25%, 30%, 35%, 40%, 45%, 50%, 55%, 60%, 65%, 70%, 75%, 80%, 85%, 90%, 95%, or 100%; or wherein the redness of the skin is reduced by an amount ranging from about 1% to 100%. In some other instances, the redness of the skin is reduced by at least about 60%, 65%, 70%, 75%, or 80%; or wherein the redness of the skin is reduced by an amount ranging from about 60% to 80%. In still other instances, the redness of the skin is reduced by at least about 70%, 75%, or 80%; or wherein the redness of the skin is reduced by an amount ranging from about 70% to 80%.

In some instances, the method improves visible or tactile texture and/or smoothness of the skin, as compared to the skin prior to step (1′), by visual and/or tactile grading. In some instances, the visible or tactile texture and/or smoothness of the skin is improved by at least about 1%, 5%, 10%, 15%, 20%, 25%, 30%, 35%, 40%, 45%, 50%, 55%, 60%, 65%, 70%, 75%, 80%, 85%, 90%, 95%, or 100%; or wherein the visible or tactile texture and/or smoothness of the skin is improved by an amount ranging from about 1% to 100%. In some other instances, the visible or tactile texture and/or smoothness of the skin is improved by at least about 30%, 35%, 40%, 45%, or 50%; or wherein the visible or tactile texture and/or smoothness of the skin is improved by an amount ranging from about 30% to 50%. In still other instances, the visible or tactile texture and/or smoothness of the skin is improved by at least about 40%, 45%, or 50%; or wherein the visible or tactile texture and/or smoothness of the skin is improved by an amount ranging from about 40% to 50%.

In some instances, the method improves hydration and/or moisturization of the skin, as compared to the skin prior to step (1′), as measured by corneometer and/or SkiCon values. In some instances, SkiCon is use to evaluate the hydration of the skin. In some instances, the hydration and/or moisturization of the skin is improved by at least about 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200%; or wherein the hydration and/or moisturization of the skin is improved by an amount ranging from about 1% to 200%. In some other instances, the hydration and/or moisturization of the skin is improved by at least about 90%, 95%, 100%, 105%, 110%, 115%, 120%, or 125%; or wherein the hydration and/or moisturization of the skin is improved by an amount ranging from about 90% to 125%. In still other instances, the hydration and/or moisturization of the skin is improved by at least about 100%, 105%, 110%, or 115%; or wherein the hydration and/or moisturization of the skin is improved by an amount ranging from about 100% to 115%.

In some instances, the method reduces transepidermal water loss by the skin and improves barrier function of the skin, as compared to the skin prior to step (1′), as measured by Tewameter values. In some instances, the transepidermal water loss by the skin is reduced by at least about 1%, 5%, 10%, 15%, 20%, 25%, 30%, 35%, 40%, 45%, or 50%; or wherein the transepidermal water loss by the skin is reduced by an amount ranging from about 1% to 50%. In some other instances, the transepidermal water loss by the skin is reduced by at least about 20%, 25%, 30%, 35%, or 40%; or wherein the transepidermal water loss by the skin is reduced by an amount ranging from about 20% to 40%. In still other instances, the transepidermal water loss by the skin is reduced by at least about 30%, 35%, or 40%; or wherein the transepidermal water loss by the skin is reduced by an amount ranging from about 30% to 40%.

In some instances, the barrier function of the skin is improved by at least about 1%, 5%, 10%, 15%, 20%, 25%, 30%, or 35%; or wherein the barrier function of the skin is improved by an amount ranging from about 1% to 35%. In some other instances, the barrier function of the skin is improved by at least about 20%, 25%, 30%, or 35%; or wherein the barrier function of the skin is improved by an amount ranging from about 20% to 35%. In still other instances, the barrier function of the skin is improved by at least about 25%, 30%, or 35%; or wherein the barrier function of the skin is improved by an amount ranging from about 25% to 35%.

In some instances, the method improves firmness and/or elasticity of the skin, as compared to the skin prior to step (1′), as measured by cutometer R2 and R0 values. In some instances, the firmness and/or elasticity of the skin is improved by at least about 1%, 5%, 10%, 15%, 20%, 25%, or 30%; or wherein the firmness and/or elasticity of the skin is improved by an amount ranging from about 1% to 30%. In some other instances, the firmness and/or elasticity of the skin is improved by at least about 15%, 20%, 25%, or 30%; or wherein the firmness and/or elasticity of the skin is improved by an amount ranging from about 15% to 30%. In still other instances, the firmness and/or elasticity of the skin is improved by at least about 20%, 25%, or 30%; or wherein the firmness and/or elasticity of the skin is improved by an amount ranging from about 20% to 30%.

In some instances, the method improves appearance of collagen in the skin, as compared to the skin prior to step (1′), as measured by Vivascope image visual grading.

In some instances, the method reduces lines and/or wrinkles on the skin, as compared to the skin prior to step (1′), by visual grading. In some instances, the lines and/or wrinkles on the skin are reduced by at least about 1%, 5%, 10%, 15%, 20%, or 25%; or wherein the lines and/or wrinkles on the skin are reduced by an amount ranging from about 1% to 25%. In some other instances, the lines and/or wrinkles on the skin are reduced by at least about 5%, 10%, 15%, or 20%; or wherein the lines and/or wrinkles on the skin are reduced by an amount ranging from about 5% to 20%. In still other instances, the lines and/or wrinkles on the skin are reduced by at least about 5%, 10%, or 15%; or wherein the lines and/or wrinkles on the skin are reduced by an amount ranging from about 5% to 15%.

For the various ranges given above, it is understood that individual values and subranges within the aforementioned ranges are also disclosed.

### EXAMPLES

### Example 1: Study I—RNA Microarray Analysis of Skin Formulations in Reconstructed Human Skin Model EPIDERM-FT™

### Materials and Methods

### Experimental Design

Gene Expression testing in EPIDERM-FT™ Skin Model:

Human 3D full-thickness skin model EPIDERM-FT™ were obtained from MatTek Corp. and acclimated in 6-well plates in vitro using standard cell culture conditions (37° C., 5% CO₂) on Day 1. Tissues were acclimated for 24 hours and randomly separated into eight (8) groups (n=3 wells). Each Treatment Group was used to test one of the test materials, referred to as Samples 1-7, or a placebo test material (Sample 8). Each of the test materials (Samples 1-7 and placebo (Sample 8)) were stored at room temperature and handled by a qualified research scientist personnel. Vials were sealed and formulations were immediately used for treatments.

The compositions of Samples 1-8 are described in Table 1 below:

TABLE 1
Composition and Concentration of Components of Samples 1-8

| | Use Level (% w/w in formula) | | | | | |
|---|---|---|---|---|---|---|
| | 1 | 1 | 1 | 1 | 0.1 | 0.1 |
| Sample # | WASTAR ™ lavender | EXO-T ™ | Ceramide Bio | UNIREPAIR ® T-43 | SFC | Bis-amino |
| 1 | X | X | X | X | — | X |
| 2 | X | X | X | X | — | — |
| 3 | X | X | X | X | X | X |
| 4 | X | X | X | X | X | — |
| 5 | X | — | — | X | X | — |
| 6 | X | — | — | X | X | X |
| 7 | X | — | — | X | — | X |
| 8 | — | — | — | — | — | — |

where “X” denotes present and “—” denotes absent

WASTAR™ Lavender, EXO-T™, UNIREPAIR® T-43, CeramideBio, and SFC were purchased from commercial sources. “Bis-amino” refers to an aqueous formulation containing approximately 20% (wt/wt) bis-amino diglycol dimaleate.

A placebo was also prepared to serve as a control formulation. The placebo included: deionized water, glycerine, distarch phosphate, ammonium acryloyldimethyltaurate/VP copolymer, SEPIMAX ZEN, SEPINOV WEO, Sesamuls OW, Sesamum indicum seed oil, malic acid, isononyl isononanoate, caprylic/capric triglyceride, tocopheryl acetate, Fision EcoSoil, and MIKROKILL COS. The placebo included no active ingredients.

All of the samples contained the same ingredients as described above for the placebo, and each Sample also contained the specific active agents listed above in Table 1. For example, Sample #3 contained water, glycerine, distarch phosphate, ammonium acryloyldimethyltaurate/VP copolymer, SEPIMAX ZEN, SEPINOV WEO, Sesamuls OW, Sesamum indicum seed oil, malic acid, isononyl isononanoate, caprylic/capric triglyceride, tocopheryl acetate, Fision EcoSoil, and MIKROKILL COS and further included active ingredients: WASTAR™ Lavender, EXO-T™1 UNIREPAIR® T-43, SFC, and Gelest XG-2820.

Tissues were dosed topically with 200 μL of test materials and incubated for 24 hours. After treatment incubation, the test material was removed, and tissues were washed with PBS. Later, total RNA was extracted from a 6 mm biopsy sample and sent to RNA Microarray facility to perform CLARIOM™ S assay for transcriptome analysis. Gene differential expression was compared to untreated control group.

Tissue assay conditions are described in Study Protocol (RNA Microarray Gene Cluster-Analysis), as detailed below.

### Tissues

EPIDERM-FT™ skin tissues were used from human neonatal (EFT-400) origin and purchased as preserved inserts from MatTek Corp. MatTek's EPIDERM-FT™ system consists of normal, human-derived epidermal keratinocytes (NHEKs) and dermal fibroblasts (HDFs), which have been cultured to form a multilayered, highly differentiated model of the human epidermis/dermis layers. These “ready-to-use” tissues are cultured on specially prepared cell culture inserts using serum-free medium. Tissues were cultured and acclimated for 24 hours before treatments and dosing procedures and tissue groups were identified using appropriate cell culture 6-well plates. All cell culture procedures were performed at the laboratory facilities of Signum Biosciences, which met standards for Biosafety level-2 (BL-2) cell culture room.

### Environmental Conditions

Tissue culture incubator was set with a consistent stream of 5% v/v CO₂ with temperature and humidity monitored, controlled, and ranged from 36° C. to 38° C. and 50% to 65%, respectively.

### Test Material Administration and Frequency

The test materials were singly administered topically using a plastic dosing (dispense) pipette of appropriate size. A fixed 200 L/well dose volume was administered to each cell culture well and incubated for 24 hours at 37° C. and 5% CO₂. After 24 hours, tissues were washed, and total RNA was isolated.

### Expression Analysis Parameters

Differential gene expression was analyzed using Transcriptome Analysis Console (TAC) Software (v4.0.1) from Thermo-Fisher Scientific Inc. Data normalization was performed using Placebo control group and parameters shown in Table 2.

TABLE 2
Analysis Information used for Gene Expression Analysis

| Analysis Setting | Parameter |
|---|---|
| Array Type | Clariom_S_Human |
| Summarization Method | Gene Level - SST-RMA |
| Pos vs Neg AUC Threshold | 0.7 |
| Genome version | Hg38 (Homo sapiens) |
| Total Gene Number | 21,448 |
| Gene-Level Fold Change | <−2 or >2 |
| Gene-Level Fold Change | <0.05 |
| ANOVA Method | Empirical Bayes |
| DABG | <0.05 |

### Results:

All RNA samples submitted to gene array passed Quality Control (QC) assessment for microarray analysis. Global gene expression profiling from 21,448 well-annotated human genes was analyzed by Affymetrix Human Clariom™ S arrays and data visualized using Transcriptome Analysis Console (TAC) Software (Thermo-Fisher Scientific, Inc). Significant expression changes between active-treated and placebo groups were filtered using fold change ≥2 (up-regulated) or ≤−2 (down-regulated) and p Value <0.05 using empirical Bayes ANOVA.

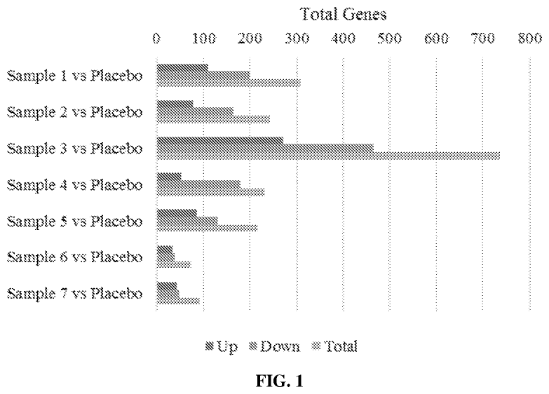

Summary results of differential gene expression between each treatment compared to the Placebo control group are shown in Table 3 below and in FIG. 1. Sample #3 produced the highest number (736) of total regulated genes (fold change ≥2 or ≤−2) with generally a down-regulation (465 genes) effect. Sample #1 produced a second tier of regulation with 309 of total regulated genes.

TABLE 3
Summary Results of Differential Gene Expression

| Comparison | Up-regulated | Down-regulated | Total |
|---|---|---|---|
| Sample #1 vs Placebo | 110 | 199 | 309 |
| Sample #2 vs Placebo | 78 | 165 | 243 |
| Sample #3 vs Placebo | 271 | 465 | 736 |
| Sample #4 vs Placebo | 53 | 179 | 232 |
| Sample #5 vs Placebo | 86 | 130 | 216 |
| Sample #6 vs Placebo | 34 | 39 | 73 |
| Sample #7 vs Placebo | 43 | 48 | 91 |

### Reinforcement of Skin Barrier Function

Both samples #3 and #4 significantly up-regulated CERS4 (ceramide synthase 4) by 42-67% compared to the placebo, suggesting a positive benefit in ceramide production for skin barrier protection. Sample #1 was the only treatment to significantly up-regulate TJP1 (tight junction protein 1, Zona Occludens 1; 108%) and OCLN (occluding; 227%), suggesting a benefit in the formation of tight junctions between cells to limit movement of substances through the paracellular space, efficient epithelial polarization and/or barrier formation. Although, not significantly, samples #1, #3 and #4 upregulated FLG (filaggrin) by 103-171%, suggesting a positive assistance in epidermal differentiation. Sample #2 was the only material to down-regulate both PTGS2 (prostaglandin-endoperoxide synthase 2; 231%) and NFKB1 (nuclear factor of kappa light polypeptide gene enhancer in B-cells 1; 110%) which are key regulators of general inflammatory mechanisms activated by intra- and extra-cellular stimuli that deteriorate the epidermal barrier.

### Cornified Envelope Development

Sample #1 significantly up-regulated small proline-rich proteins (SPRR2G=631%; SPRR3=726%), which results in the formation of the late-differentiated keratinocyte envelope beneath the plasma membrane. Sample #3 up-regulated SPRR4 (small proline-rich protein 4) that is a cross-linked envelope protein of keratinocytes involved in UV-induced cornification. Although not significantly, sample #1 also up-regulated LOR (loricrin, 250%), which is a major protein component of the cornified cell envelope.

### Cornified Envelope Lipid Synthesis

Sample #3 significantly down-regulated UGCG (UDP-glucose ceramide glucosyltransferase; −163%) and FASN (fatty acid synthase; −33%) which are involved in the de novo biosynthesis of glycosphingolipids and long-chain saturated fatty acids, respectively, which are part of the cornified envelope in terminally keratinocytes. Samples #1 and #3 were the best materials to up-regulate (40-128%) genes of the late cornified envelope mechanism (LCE1A, LCE1B, LCE1D, LCE1F, LCE2C, LCE2D, LCE6A). In contrast, samples #5-7 down-regulated some of these late cornified envelope genes (LCE3E, LCE5A), thus producing a negative effect in this mechanism.

### Example 2: Study II—RNA Microarray Analysis of Skin Formulations in Reconstructed Human Skin Model EPIDERM-FT™

### Materials and Methods

### Study Details

A second study was performed by Signum Biosciences, Inc. using the same procedure as described in Example 1 and the Study Protocol, below, however, different sample formulations were tested. In addition to testing the same placebo as tested in Example 1, untreated tissue was also tested as a control. RNA samples were sent for microarray assay (see details in Study Protocol below). Transcriptome profiling results were obtained one week later.

Each Treatment Group was used to test one of the test materials, referred to as Samples 1-6, or a placebo test material or was left untreated. Each of the test materials (Samples 1-6 and placebo) were stored at room temperature and handled by a qualified research scientist personnel. Vials were sealed and formulations were immediately used for treatments.

The compositions of Samples 1-6 are described in Table 4 below:

TABLE 4
Composition and Concentration of Components of Samples #1-6

| | Use Level (% w/w in formula) | | | | |
|---|---|---|---|---|---|
| | 1 | 1 | 1 | 1 | |
| | Wastar ™ | EXO- | Ceramide | UNIREPAIR ® | 0.1 |
| Sample # | lavender | T ™ | Bio | T-43 | SFC |
| 1 | X | X | — | — | — |
| 2 | X | — | X | — | — |
| 3 | X | — | — | X | — |
| 4 | X | — | — | — | X |
| 5 | — | X | — | X | — |
| 6 | — | — | X | X | — |

where “X” denotes present and “—” denotes absent

In addition to Samples #1-6, listed above, an “Untreated” Group and a “Placebo” Group were evaluated. Samples #1-6 were prepared in a similar manner to Sample #3 of Example 1 described above. A “placebo” was also prepared which is the same as described in Example 1 above.

### Results:

All RNA samples submitted to gene array passed Quality Control (QC) assessment for microarray analysis.

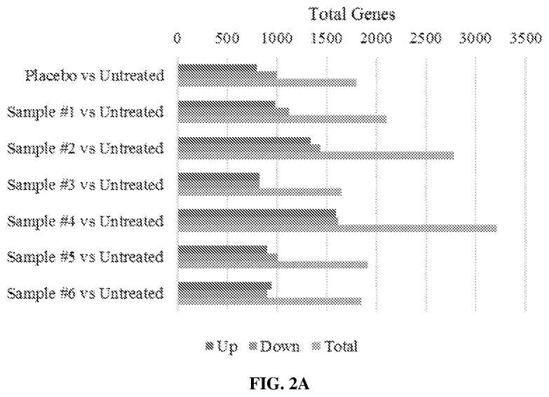

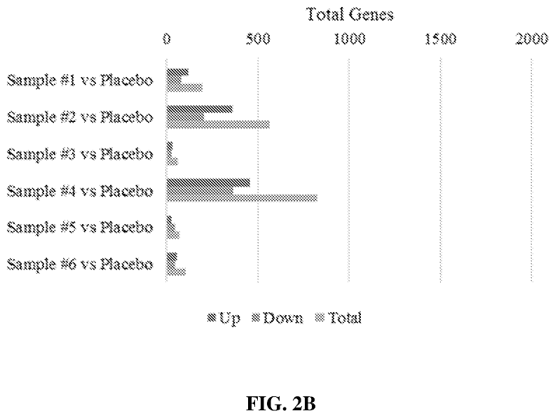

Summary results of differential gene expression between each treatment compared to untreated or Placebo control groups are shown in Table5 and in FIGS. 2A and 2B. Treatments with sample #4 was the material that produced the highest number (822) of total regulated genes (fold change ≥2 or ≤−2) with generally an up-regulation (457 genes) effect. Sample #2 modulated the second most with 564 total regulated genes.

TABLE 5
Summary results of differential gene expression

| Comparison | Up-regulated | Down-regulated | Total |
|---|---|---|---|
| Sample #1 vs Untreated | 982 | 1119 | 2101 |
| Sample #2 vs Untreated | 1342 | 1438 | 2780 |
| Sample #3 vs Untreated | 820 | 828 | 1648 |
| Sample #4 vs Untreated | 1593 | 1612 | 3205 |
| Sample #5 vs Untreated | 905 | 1004 | 1909 |
| Sample #6 vs Untreated | 949 | 900 | 1849 |
| Sample #1 vs Placebo | 120 | 78 | 198 |
| Sample #2 vs Placebo | 358 | 206 | 564 |
| Sample #3 vs Placebo | 31 | 28 | 59 |
| Sample #4 vs Placebo | 457 | 365 | 822 |
| Sample #5 vs Placebo | 28 | 44 | 72 |
| Sample #6 vs Placebo | 54 | 48 | 102 |

### Reinforcement of Skin Barrier Function

The placebo significantly down-regulated ceramide synthases 2 and 3 (CERS2, CERS3) compared to untreated. Samples #4, #5 and #6 significantly up-regulated CERS5 (ceramide synthase 5) by 74-102% compared to placebo, with sample #4 showing the strongest effect (97%) suggesting a positive benefit in ceramide production for skin barrier protection. The placebo also significantly down-regulated TJP1 (tight junction protein 1, Zona Occludens 1) and occluding (OCLN) compared to untreated and none of the treatments significantly up-regulated these genes compared to placebo. Although, not significantly compared to placebo, samples #1, #3, #5 and #6 upregulated FLG (filaggrin) by 32-636%, thus suggesting a positive assistance in epidermal differentiation. The placebo up-regulated both PTGS2 (prostaglandin-endoperoxide synthase 2) and NFKB1 (nuclear factor of kappa light polypeptide gene enhancer in B-cells 1) compared to untreated. Sample #2 was the only material to down-regulate compared to placebo both PTGS2 (−20%) but not NFKB1 which are key regulators of general inflammatory mechanisms activated by intra- and extra-cellular stimuli that deteriorate the epidermal barrier.

### Cornified Envelope Development

The placebo significantly down-regulated Small Proline-Rich Protein genes (SPRR2B, SPRR2D, SPRR2E, SPRR2F, SPRR2G, SPRR4) compared to untreated. None of the treatments significantly up-regulated small proline-rich proteins compared to placebo, but sample #1 upregulated SPRR2A, SPRR2B, SPRR2D, SPRR2E, SPRR2F, SPRR2G and SPRR4 by 19-219%, which results in the formation of the late-differentiated keratinocyte envelope beneath the plasma membrane. The placebo also significantly down-regulated LOR (loricrin) compared to untreated. Although not significantly, samples #1, #3, #5, and #6 also up-regulated LOR by 35%-563% compared to the placebo, which is a positive effect since loricrin is a major protein component of the cornified cell envelope.

### Cornified Envelope Lipid Synthesis

The placebo did not significantly up-regulate UGCG (UDP-glucose ceramide glucosyltransferase) or FASN (fatty acid synthase), which are involved in the de novo biosynthesis of glycosphingolipids and long-chain saturated fatty acids, respectively, which are part of the cornified envelope in terminally keratinocytes. However, sample #3 and #5 significantly up-regulated UGCG by 66-67% compared to placebo. The placebo significantly down-regulated all the genes in late cornified envelope (LCEs) mechanism compared to untreated and none of the sample treatments significantly up-regulated genes of the late cornified envelope (LCEs) mechanism compared to placebo, but samples #1-#4 demonstrated up-regulation of some of these genes (LCE3B, LCE5A). All treatments significantly down-regulated compared to placebo some of these late cornified envelope genes, thus producing a negative effect in this mechanism.

### Study Protocol for Examples 1 and 2

### RNA Microarray Gene Cluster-Analysis

TISSUES: Full Thickness Reconstructed Human Skin (EPIDERM-FT™) model were purchased from MatTek Corp. (Cat #EFT-400; Lot #34296, Kit P, Q)

TISSUE CULTURE: EpiDerm-FT™ model will be cultured in serum-free medium from MatTek Corp. (Cat #EFT-400-ASY; Lot #101722GSA) at 37° C. with 5% CO₂. Skin tissues derived from neonatal donors were obtained in 6-well plate format and acclimated for 24 hours.

DOSE ROUTE/FREQUENCY: Single topical administration for 24 hours at 37° C. and 5% CO₂.

GLP COMPLIANCE: Non-GLP

### Protocol:

### Day 1: Tissue Equilibration

a) Obtained EPIDERM-FT™ tissue inserts from MatTek Corp. and followed manufacture's protocol for tissue equilibration.
b) Transferred tissue inserts into 6-well plates pre-filled with 2.5 mL media per well.
c) Incubated for 24 hours at 37° C. and 5% CO₂.

### Day 2: Treatments

a) After tissue equilibration period, transferred tissue inserts into fresh 6-well plates pre-filled with 2.5 mL media per well.
b) Dosed tissues in the basal compartment (200 μL topically) drop-wise using a positive displacement micropipette with each treatment (n=3).
c) Incubated tissues for 24 hours at 37° C. and 5% CO₂.

### Day 3: RNA Extraction

Reagent: RNAQUEOUS® kit (ThermoFisher, Cat No. AM1912)
a) After 24-hours incubation with test materials, washed tissues with sterile 1×DPBS and removed remaining wash solution. Removed any excess of DPBS from the tissue by gently shaking the insert and blotting it on the sterile blotting paper.
b) Removed tissues from inserts using forceps and obtained a 6 mm punch biopsy per tissue and added 450 μL of Lysis/Binding solution into a 1 mL homogenization vessel (Kontes Duall ground glass tissue grinder).
c) Dropped tissues into the Lysis/Binding solution and homogenized until formation of homogenous lysate (~10 strokes).
d) Transferred lysate to a labeled-fresh RNase-free microcentrifuge tube and incubated on ice.
e) Heated a 2.5 mL aliquot of Elution Solution in an RNase-free microcentrifuge tube in a heat block set to 70-80° C.
f) Mixed 350 μL of 64% Ethanol with 350 μL of tissue lysate in a labeled-fresh RNase-free microcentrifuge and mixed gently but thoroughly by vortexing.
g) Applied the lysate/ethanol mixture (from the previous step) to a Filter Cartridge assembled in a Collection Tube (supplied) and centrifuged for 1 min at 10,000-14,000 rcf
h) Discarded the flow-through and reused the Collection Tube for the washing steps.
i) Applied 700 μL Wash Solution #1 to the Filter Cartridge. Added the washes through the filter as in the previous step. Discarded the flow-through and reused the tube for subsequent washes.
j) Added 500 μL Wash Solution #2/3. Added the wash solution through the filter as in the previous step.
k) Repeated with a second 500 μL aliquot of Wash Solution #2/3.
l) After discarding the wash solution, continued centrifugation to remove the last traces of wash solution.
m) Putted the Filter Cartridge into a fresh Collection Tube.
n) Pipetted 50 μL Elution Solution preheated to ~70-80° C. to the center of the filter. Closed the cap of the tube. Recovered eluate by centrifugation for ~30 seconds at room temperature (RCF 10,000-14,000×g).

### RNA Microarray Analysis

RNA samples were submitted for Expression Profiling services using Affymetrix Human CLARIOM™ S array (21,448 coding genes). Human Gene Cluster analysis was be performed focusing on well-annotated genes to perform gene-level expression profiling and assess changes in key genes and pathways. Gene differential expression was quantified using CLARIOM™ S microarray method after twenty-four (24) hour incubation with test materials exposure.

Global gene expression profiling from 21,448 well-annotated human genes was analyzed by Affymetrix Human CLARIOM™ S arrays. Significant expression changes were filtered using fold change based on untreated group: >2 (up-regulated) or <−2 (down-regulated).

### Example 3: Clinical Study on Skin Formulations

In this study, the efficacy of a skin formulation, denoted as Face Serum product below, on 40 healthy subjects with very dry skin/very damaged skin of all skin types was evaluated. The study subjects' skin was evaluated using expert Visual/Tactile Grading, Corneometer, TEWL, Cutometer, VivaScope, SkiCon and Self-Perception Questionnaire over three months/90 days of use at home, when following the directions of usage provided to study participants. This was a single center, at home-use study.

### Selection of Study Subjects:

### Screening:

32 females and 8 males were enrolled as subjects of the study so that a minimum of 30 subjects would be expected to complete the study. Subjects satisfied the following inclusion and exclusion criteria, were prepared to accept the prohibitions and restrictions, and gave written informed consent.

The suitability of each subject to participate was confirmed prior to their acceptance into the study by completion and review of a study-specific pre-study questionnaire.

### Methods

Ivory Soap washout product was supplied to the study participants. The Face Serum product was certified as having been manufactured/formulated with ingredients that are safe and suitable for the product's stated purpose.

The Face Serum product contained the following ingredients: Aqua, Sodium Benzoate, Niacinamide, Lactic Acid, Hydrogenated Ethylhexyl Olivate, Hydrolyzed Vegetable Protein, Glycerin, Sodium Hydroxide, Lactococcus Ferment Lysate, Adenosine Triphosphate, Camellia japonica Seed Oil, Sodium Hyaluronate, Polyglyceryl-3 Cetyl Ether, Polyglyceryl-4 Caprate, Hydroxyapatite, Ceramide Ap, Isononyl Isononanoate, Phytosphingosine, Caprylic/Capric Triglyceride, Bis-Aminopropyl Diglycol Dimaleate, Cetylhydroxyproline Palmitamide, Sodium Acetylated Hyaluronate, Hydrogenated Olive Oil Unsaponifiables, Tetraacetylphytosphingosine, Propanediol, Sodium Lactate, Sesamum indicum (Sesame) Seed Oil, Sodium Chloride, Caprylyl Glycol, Sorbitan Isostearate, Acetyl Tyrosine, Vibrio alginolyticus Ferment Filtrate, 1,2-Hexanediol, Sodium Hyaluronate Crosspolymer, Hydrolyzed Sodium Hyaluronate, Xanthan Gum, Disodium Succinoyl Famesylcysteine, Tocopherol, Cetearyl Alcohol, Tetrasodium Glutamate Diacetate, Malic Acid, Zingiber officinale (Ginger) Root Extract, Bisabolol, Citric Acid, Ethylhexylglycerin, Trisodium Citrate, Proline, Ceramide Eop, Tocopheryl Acetate, Glycosphingolipids, Hydrogenated Lecithin, Hydrogenated Polydecene, Palmitoyl Tripeptide-1, Hydroxyacetophenone, Palmitoyl Tetrapeptide-7, Pentylene Glycol, Ceramide Np, Butylene Glycol, Beta-Glucan, Sodium Polyacryloyldimethyl Taurate, Stearic Acid, Glyceryl Stearate, Cholesterol, Hydroxyethyl Acrylate/Sodium Acryloyldimethyl Taurate Copolymer, Lavandula angustifolia Flower/Leaf/Stem Extract, and Polyacrylate Crosspolymer-6.

The Face Serum product (or test article) was used as supplied, following usage instructions, as follows: Apply a generous amount of product on face and forearm, one to two pumps, after cleansing/toner, twice a day (morning and night) prior to applying your regular regimen. No other serums/moisturizers should be used. For each day, indicate whether you used the product. Product should be used twice daily for the 5 continuous days prior to visit 2 and 90 days thereafter.

### Study Procedure:

### Visit 1—Washout (Day-5):

Subjects meeting enrollment criteria were provided with a washout product, to replace their usual facial cleansers and to be used daily for 5 continuous days on the face and forearms prior to study visit 2. A usage diary was issued to subjects to track the washout progress. Instructions were given to subjects not to apply any creams or moisturizers to the face or forearms starting from 2 days prior to visit 2 and, for the entire duration of the study. Subjects were also instructed to come to all study visits without makeup. Subjects also brought along their diary and washout product to the next visit and washed their face 2.5 hours prior to their visit.

### Visit 2—Baseline (Study Day 1):

Subjects attended the testing facility where eligibility was verified, and diaries checked, and washout product collected. Once accepted into the study, subjects acclimatised to indoor ambient conditions, temperature 20-22° C. and relative humidity 30-50%, for at least 30 minutes before assessments. Baseline measurements were then taken, where the subject's face was assessed visually and tactilely by an expert grader. Corneometer, SkiCon and TEWL assessments were also assessed using the forearm, while Vivascope, and Cutometer assessments were conducted on the face.

Once baseline assessments were completed, the test article was applied to the face and the forearm after which expert visual assessments of the face and Corneometer, SkiCon and TEWL assessments of the forearm were reassessed. Subjects also completed a Self-perception Questionnaire (SPQ). Subjects were then issued with the product and usage instructions to take home and use as instructed for the next 3 months. Subjects also ensured that their face was washed at least 2.5 hours prior to each visit.

### Visit 3—Study Day 7:

Subjects returned to the testing facility after using the product (as instructed) at home for 7 days. Test articles were weighed, and diaries were checked for compliance. Subjects also acclimatized to indoor ambient conditions as described above in the Baseline test (Study Day 1) and the subject's face was then visually and tactilely assessed by an expert grader and Corneometer, SkiCon and TEWL assessments were made on the forearm, and Cutometer assessments were conducted on the face. Subjects also completed a self-perception questionnaire.

### Visit 4—Study Day 30:

Subjects returned to the testing facility after using the product as instructed at home for 30 days. The same measurements were taken and inspections were conducted as in Visit 3.

### Visit 5—Study Day 60:

Subjects returned to the testing facility after using the product as instructed at home for 60 days.

The same measurements were taken and inspections were conducted as in Visit 3.

Additionally, VivaScope assessments were conducted on the face.

### Visit 6—Study Day 90 (Final Visit):

Subjects returned to the testing facility after using the product as instructed at home for 90 days.

The same measurements were taken and inspections were conducted as in Visit 5.

Upon completion of all assessments and receipt of all diaries and test articles, subjects' participation was considered complete.

### Visual Assessment Via Visual and Tactile Grading

Visual assessments were conducted on the face at baseline, after immediate use (1 hour), Day-7, Day-30, Day-60, and Day-90 while tactile assessment was conducted on the face at baseline, Day-7, Day-30, Day-60, and Day-90. The same Expert grader assessed all subjects for the duration of the study according to the grading scales below. Illumination of the subject's face was by a 60-watt pearl bulb, approximately 30 cm from the site. The PCR Clinical Grading Scale used in the study is shown in FIG. 3A.

### Instrumental Assessments

### Corneometer

Moisturization measurements were performed using the Corneometer CM825 (Courage and Khazaka, Germany). This instrument relied on the dielectric constant, a physical property of water, which is relatively high and as such affects the capacitance of a capacitor. Any change in the dielectric constant due to skin moisture variations alters the capacitance of the precision capacitor in the instrument. These variations are detected electronically and are converted into a value by the Corneometer.

Triplicate readings were made using the probe attachment of the Corneometer at the test site on the forearm. Between each assessment the probe attachment of the Corneometer was pressed onto a dry tissue. Subjects were in the controlled environment (at a temperature of 20-22° C. and at a relative humidity of 30-50%) for at least 15 minutes prior to any assessments being performed. If one measurement was more than ±10 units (outlier) from the other measurements, a fourth measurement was taken. Measurements were taken at baseline, after immediate use (1 hour), Day-7, Day-30, Day-60, and Day-90.

### SkiCon

Skin conductance can be used to measure skin moisture levels derived from alterations in the electrical properties of the stratum corneum.

SkiCon 200EX (IBS co. Hamamatsu, Japan) measures skin conductance through the stratum corneum at a fixed high frequency current of 3.5 MHz. The apparatus gives readings of conductance in siemens (μS), ranging from 0-1999. All conductance measurements are given after a reading of 3 s. Measurements were taken from the forearm at baseline, after immediate use (1 hour), Day-7, Day-30, Day-60, and Day-90.

Trans epidermal water loss (TEWL) by Tewameter® Probe

Trans epidermal water loss was be measured with the Tewameter® TM300 Probe (Courage and Khazaka; Koln, Germany). The measurement of the water evaporation and therefore TEWL, is based on the diffusion principle in an open chamber and is measured as g/m²/h. The density gradient is measured indirectly by two pairs of sensors in the probe attachment, one for temperature and the other for relative humidity. This density gradient was then analyzed by a microprocessor in the instrument. Measurements were taken from the forearm at baseline, after immediate use (1 hour), Day-7, Day-30, Day-60, and Day-90.

VIVASCOPE® 1500—Assessment of collagen:

For Vivascope assessment, subjects were in a controlled environment (at a temperature of between 20° C.-24° C. and relative humidity of 35%-50%) for 30 minutes prior to any assessments being performed. Measurements were taken from the right crow's feet area at baseline, Day-60, and Day-90.

The test area was captured with a VivaCam, which allowed the confocal and photographic images to be correlated.

The collagen structure (i.e., the fragmentation and density of collagen in the papillary and superficial dermis) was visualized in vivo by confocal laser scanning microscopy (VIVASCOPE® 1500; VivaScope GmbH, Munich, Germany). This allowed the cellular microstructure to be visualized as optical cross sections with a horizontal resolution of up to 1.25 μm and vertical resolution of 3-5 μm, up to a maximum depth of the superficial dermis (200 μM). Collagen fibers appeared as white structures.

Individual images (500 μm×500 μm) were taken (approx. 40) at 5 μm scanning steps to create a stack of image. Only the images targeting the collagen network (in the papillary and superficial dermis) were analyzed. Images taken at baseline were compared to those taken at Day-60 and separately to those at Day-90. The images for each were graded for collagen fragmentation and density by an expert grader on a visual analog scale. The PCR Clinical Grading Scale used for Vivascope assessment of collagen is provided in FIG. 3B.

### Firmness (R0)/Elasticity (R2) by Cutometer®:

The viscoelastic properties of the skin (elasticity and firmness) was performed using the Cutometer® MPA 580 (Courage and Khazaka, Germany) at baseline, Day-7, and Day-90.

The measurements for passive stretching were recorded. This measurement was displayed as curves at the end of each measurement using Windows® based software.

Measuring mode 1 was used with constant suction of 400 mbar for five seconds followed by relaxation time of 3 seconds with three repetitions. Elasticity parameters measured were R2 and R0 which serves an indication of firmness, an average of the three repetitions were reported.

### Self-Perception Questionnaire:

Study subjects also completed a self-perception questionnaire on Day-1 (Immediately after use, up to 1 hour), Day-7, Day-30, Day-60, and Day-90.

### Study Results:

Forty subjects were recruited into, and 38 subjects completed the study. Two subjects did not complete the study and withdrew for personal reasons. The table below provides details of the study subjects.

TABLE 6
Information on Study Subjects
Age

| | |
|---|---|
| Count | 40 |
| Mean | 41.50 |
| Std | 13.69 |
| Median | 37.50 |
| Range | 24-65 |

Race

| | |
|---|---|
| White | 29 (72.5%) |
| Black | 6 (15%) |
| Asian | 2 (5%) |
| Biracial | 2 (5%) |
| Indigenous | 1 (2.5%) |

Fitzpatrick

| | |
|---|---|
| II | 19 (47.5%) |
| III | 7 (17.5%) |
| I | 6 (15%) |
| IV | 4 (10%) |
| V | 4 (10%) |

Gender

| | |
|---|---|
| Female | 32 (80%) |
| Male | 8 (20%) |

No adverse reactions were reported. The study protocol was followed with minor deviations in the demographics, which did not affect the integrity of the study.

### Conclusion

### Visual and Tactile Grading:

For all visual grading assessments, a decrease in mean score indicates an improvement in the respective assessment. Under the conditions of the study, the test article performed statistically significantly at reducing the mean scores for all visual grading.

Within treatment analysis of visual grading scores for medium/deep lines wrinkles of the face showed a statistically significant decrease in mean scores post application by Day 90, when compared to baseline. Additionally, there was a sustained percentage decrease from baseline for the 1 hour, Day 7, Day 30 and Day 60 timepoints, and a statistically significant mean percent decrease in the appearance of wrinkles after 90 days of test article use, twice daily. Therefore, the test article contributed to a decrease in the appearance of medium/deep lines wrinkles after 90 days of use.

Within treatment analysis of visual grading scores for skin radiance/luminosity of the face showed a statistically significant decrease in mean scores post application when compared to baseline. The mean score at baseline decreased post application at 1 hour, Day 7, Day 30, Day 60, and Day 90 respectively. Hence, there was a statistically significant mean percent change (increase in skin radiance appearance to Total brightness, translucent appearance) at all timepoints after 90 days of use, twice daily.

Within treatment analysis of visual grading of skin redness of the face also showed a statistically significant decrease in mean scores post application compared to baseline. The mean score at baseline decreased post application at 1 hour, Day 7, Day 30, Day 60, and Day 90 respectively. Hence, there was a statistically significant mean percent change (decrease in skin redness appearance to no redness) at all timepoints after 90 days of use, twice daily.

Within treatment analysis of visual grading scores for skin texture/smoothness of the face showed a statistically significant decrease in mean scores post application, when compared to baseline. The mean score at baseline decreased post application at 1 hour, Day 7, Day 30, Day 60, and Day 90 respectively. Hence, there was a statistically significant mean percent change (increase in skin texture/smoothness appearance to smooth, soft, even looking skin texture) at all timepoints after 90 days of use, twice daily.

Within treatment analysis of tactile grading of skin texture/smoothness of the face also showed a statistically significant decrease in mean scores post application when compared to baseline. The mean score at baseline decreased post application at 1 hour, Day 7, Day 30, Day 60, and Day 90 respectively. Hence, there was a statistically significant mean percent change (increase in skin texture/smoothness to smooth, soft, even skin texture) at all timepoints after 90 days of use, twice daily.

### Corneometer & SkiCon for Hydration/Moisturization:

Under the conditions of the study, the test article performed statistically significantly at improving skin hydration/moisture as measured by Corneometer and SkiCon values at all timepoints. A within-treatment analysis showed data from 1 hour, Day 7, Day 30, Day 60, and Day 90 were significantly improved when compared to baseline readings. Notably, for Corneometer measurements, there was an increase in hydration of skin in an hour and a change from baseline after 90 days of test article use, twice daily. This coincided with increases in SkiCon measurements with an increase in an hour and a change from baseline after 90 days of test article use, twice daily.

### Tewameter for Trans Epidermal Water Loss (TEWL):

Under the conditions of the study, the test article also performed statistically significantly at reducing trans-epidermal water loss as measured by Tewameter® values at all timepoints (p<0.01). A within-treatment analysis showed data from 1 hour, Day 7, Day 30, Day 60, and Day 90 were significantly reduced when compared to baseline readings. There was progressive percentage reduction in trans epidermal water loss (increase in barrier function) after 90 days of test article use, twice daily.

### Cutometer R0/R2 for Firmness and Elasticity:

Under the conditions of the study, the test article performed statistically significantly at increasing skin elasticity as measured by Cutometer R2 values (p<0.01). A within-treatment analysis showed data from 1 hour, Day 7, Day 30, Day 60, and Day 90 were significantly improved when compared to baseline readings. There was progressive percentage increase in skin elasticity after 90 days of test article use, twice daily.

Under the conditions of the study, the test article performed statistically significantly at improving skin firmness as measured by Cutometer R0 values. A within-treatment analysis showed data from 1 hour, Day 7, Day 30, Day 60, and Day 90 were significantly improved when compared to baseline readings. There was progressive percentage increase in skin firmness after 90 days of test article use, twice daily.

### Vivascope (Visual)—Collagen:

Data from the visual grading for collagen were taken from the right crow's feet area. Vivascope images showed that the test article performed statistically significantly at improving the appearance of collagen, as measured by Vivascope image visual grading (p<0.05). Under the conditions of the study, a within-treatment analysis showed mean data from Day 60 and Day 90 were statistically significantly decreased when compared to baseline readings (p<0.05).

### Self-Perception Questionnaire:

Subjects completed Self-Perception Questionnaires immediately after first application of the test article and after 7, 30, 60 and 90 days of twice daily product use. Subjects used a 5-Point Likert Scale to indicate how strongly they agreed/disagreed to statements about the test article and the subject's experience with the test product. Data from the self-perception questionnaires showed that the test article performed either favorable (>50%) or highly favorable (>80%) in all questions asked at all time points.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific instances of the invention described herein. Such equivalents are intended to be encompassed by the following claims.

We claim:

1. A skin formulation comprising:
an active agent which is
(i) 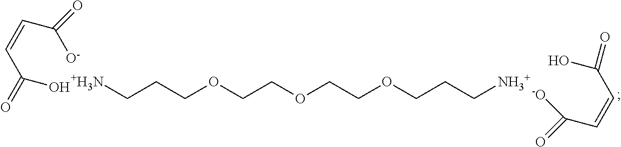
(ii) a first composition comprising glycerin and Lavandula angustifolia extract;
(iii) a second composition comprising Vibrio alginolyticus ferment filtrate;
(iv) optionally a third composition comprising acetyl tyrosine, proline, and adenosine triphosphate;
(v) cetylhydroxyproline palmitamide;
(vi) optionally N-Succinyl-S-Farnesyl-L-Cysteine;
(vii) optionally one or more dermatologically acceptable excipients; and
(viii) one or more dermatologically acceptable carriers.

2. The skin formulation of claim 1, wherein the second composition further comprises butylene glycol.

3. The skin formulation of claim 1, wherein the third composition further comprises butylene glycol and hydrolysed vegetable protein.

4. The skin formulation of claim 1, wherein the active agent is present in an amount ranging from about 0.001 wt % to about 10 wt % of the total weight of the skin formulation.

5. The skin formulation of claim 1, wherein the active agent is present in an amount ranging from about 0.001 wt % to about 5 wt % of the total weight of the skin formulation.

6. The skin formulation of claim 1, wherein the first composition, the second composition, the third composition, the cetylhydroxyproline palmitamide, and the N-Succinyl-S-Farnesyl-L-Cysteine are each independently present in an amount ranging from about 0.001 wt % to about 5 wt % of the total weight of the skin formulation.

7. The skin formulation of claim 1, wherein the first composition, the second composition, the third composition, and the cetylhydroxyproline palmitamide, are each independently present in an amount ranging from about 0.001 wt % to about 5 wt % of the total weight of the skin formulation.

8. The skin formulation of claim 1, wherein the one or more dermatologically acceptable excipients are present and selected from the group consisting of an abrasive, an acidulent, an adhesion promoter, an antioxidant, an antiseptic, an astringent, a barrier agent, a bio-adhesive agent, a botanical agent, a buffer agent, a bulking agent, a calming agent, a chelating agent, a cleansing agent, an emulsifier agent, a colorant, a conditioning agent, a cooling agent, a deodorant agent, a depilatory agent, a detergent, a disinfectant, a dispersant, an emollient, an emulsifier, an emulsion stabilizer, an essential oil, an exfoliant, a fat soluble agent, a fiber, a film former, a fixative, a foam booster, a foam stabilizer, a foaming agent, a fragrance, a free radical scavenger, a gellant, a glosser, a hair growth promoter, a conditioning agent, a humectant, a lubricant, a make-up agent, a moisture barrier agent, a moisturizing agent, a neutralizer, an odor-masking agent, an oil, an oil absorbent agent, an opacifier, an oxidant, a perfume, a perfume solvent, a perfume stabilizer, a pigment, a plant extract, a plant derivative, a plant tissue extract, a plant root extract, a plant seed extract, a plant oil, a plasticizer, a polymer film former, a preservative agent, a propellant, a re-fatting agent, a sequestrant, a silicone agent, a skin clarifier, a skin cleanser, a skin lipid, a skin lightening agent, a solubilizer, a suspending agent, a sun protection agent, a soothing agent, a stabilizer, a sunscreen, a sunscreen UVA, a sunscreen UVB, a broad-band sunscreen, a surfactant, an amphoteric surfactant, an anionic surfactant, a cationic surfactant, a non-ionic surfactant, a silicone surfactant, a thickening agent, a tightening agent, a toner, a tonic agent, a viscosity stabilizer, a vitamin, a water proofing agent, a wax, a wetting agent, and combinations thereof.

9. The skin formulation of claim 1, the one or more dermatologically acceptable excipients are present and wherein the total weight percentage of the one or more dermatologically acceptable excipients in the skin formulation is in an amount ranging from about 0.001 wt % to about 99 wt % of the total weight of the skin formulation.

10. The skin formulation of claim 1, wherein the skin composition further comprises one or more additives selected from the group consisting of an agent modulating cell differentiation, an agent modulating cell proliferation, an agent stimulating synthesis of dermal or epidermal macromolecules, an agent preventing degradation of dermal or epidermal macromolecules, an agent acting on microcirculation, an antimicrobial sequestering agent, an anti-acne agent, an anti-aging agent, an anti-wrinkle agent, an anti-atrophy agent, an anti-bacterial agent, an anti-scar agent, an anti-inflammatory agent, an anti-irritant agent, an anti-microbial agent, a collagen stimulating agent, an elastin stimulating agent, an extracellular matrix stimulating agent, an enzyme, an enzymatic inhibitor, an enzyme-inducing agent, a hyaluronic acid stimulating agent, a keratolytic agent, a natural moisturizing agent, a peptide agent, a protein agent, a rosacea inhibitory agent, a skin barrier agent, a skin barrier restoration agent, a skin calming agent, a skin smoothing agent, a skin calming agent, a skin soothing agent, a skin penetration enhancing agent, a skin penetration retarding agent, a skin conditioning agent, a skin exfoliating agent, a skin peeling agent, a skin healing agent, a skin protectant agent, a skin purifier agent, a wound healing agent, and combinations thereof.

11. The skin formulation of claim 1, wherein the skin composition further comprises one or more additives and wherein the total weight percentage of the one or more additives in the skin formulation is in an amount ranging from about 0.001 wt % to about 99 wt % of the total weight of the skin formulation.

12. The skin formulation of claim 1, wherein the one or more dermatologically acceptable carriers are selected from the group consisting of water, dermatologically acceptable organic solvents, and combinations thereof.

13. The skin formulation of claim 12, wherein the total weight percentage of the one or more dermatologically acceptable carriers in the skin formulation is in an amount ranging from about 0.01 wt % to about 95 wt % of the total weight of the skin formulation.

14. The skin formulation of claim 1, wherein the skin formulation comprises the third composition and the N-Succinyl-S-Farnesyl-L-Cysteine and the skin formulation is a serum.

15. The skin formulation of claim 1, wherein the skin formulation is a moisturizer.

16. A method of treating skin, the method comprising:
(i) topically applying the skin formulation of claim 1 to the skin of a subject.

17. The method of claim 16, wherein following step (i), the skin formulation remains on the skin of the subject for a period of time up to 24 hours, up to 20 hours, up to 15 hours, up to 10 hours, up to 8 hours, up to 7 hours, up to 6 hours, up to 5 hours, up to 4 hours, up to 3 hours, up to 2 hours, or up to 1 hour.

18. The method of claim 16, further comprising repeating step (i) once a day so that each day the formulation is applied two times, over a period of time of 1 day to 6 months, optionally wherein the skin formulation is applied onto the skin of the subject for a period of time of at least about 1 day, 2 days, 3 day, 4 days, 5 days, 6 days, 7 days, two, weeks, three weeks, four weeks, one month, two months, three months, four months, five months, or six months.

19. The method of claim 18, wherein a skin rejuvenation effect and/or an improvement in appearance of the skin is observed, when evaluated by visual inspection after the period of time, as compared to the skin prior to step (i).

20. The method of claim 18, wherein an effect of maintaining healthy skin; restoring damaged skin; enhancing the restoration of skin after a cosmetic and/or dermatological procedure; enhancing wound healing; or a combination thereof, is observed in the skin when evaluated by visual inspection after the period of time, as compared to the skin prior to step (i).

21. The method of claim 18, wherein one or more visible signs of aging of the skin are reduced when evaluated by visual inspection after the period of time, as compared to the skin prior to step (i), and
wherein the signs of aging are selected from the group consisting of fine lines, medium lines, deep lines, wrinkles, enlarged pores, roughness, dryness, redness, loss of elasticity, loss of volume, atrophic skin, stretch marks, puffy eyes, and dark under eye circles, and combinations thereof.

22. The method of claim 18, wherein the skin's barrier function, cornified envelope development, and/or cornified envelope lipid synthesis of the skin is improved, after the period of time, when compared to the skin prior to step (i), as demonstrated by gene expression analysis.

23. The method of claim 18, wherein the radiance, brilliance, and/or luminosity of the skin is improved when evaluated by visual grading after the period of time, as compared to the skin prior to step (i).

24. The method of claim 18, wherein the visible texture, tactile texture and/or smoothness of the skin is improved when evaluated by visual and/or tactile grading after the period of time, as compared to the skin prior to step (i).

25. The method of claim 18, wherein the hydration and/or moisturization of the skin is improved after the period of time, as compared to the skin prior to step (i).

26. The method of claim 18, wherein the firmness and/or elasticity of the skin is improved after the period of time, as compared to the skin prior to step (i).

27. The method of claim 18, wherein appearance of collagen in the skin is improved after the period of time, as compared to the skin prior to step (i).

28. The method of claim 18, wherein lines and/or wrinkles on the skin are reduced after the period of time, as compared to the skin prior to step (i), when measured by visual grading.

29. The method of claim 18, wherein the skin formulation comprises the third composition and the N-Succinyl-S-Famesyl-L-Cysteine and the skin formulation is a serum, and
wherein barrier function, brilliance, hydration, smoothness, firmness, elasticity, texture, and/or luminosity of the skin is improved after the period of time, as compared to the skin prior to step (i); and/or
wherein redness, lines, and/or wrinkles of the skin reduced after the period of time, as compared to the skin prior to step (i).

30. The method of claim 16, wherein the skin formulation is a moisturizer.
